(19) United States
(12) Patent Application Publication
(10) Pub. No.: US 2016/0272709 A1
(43) Pub. Date: Sep. 22, 2016

(54) ANTI-KIR ANTIBODIES

(71) Applicant: The Regents of the University of Michigan, Ann Arbor, MI (US)

(72) Inventor: Bruce Richardson, Ann Arbor, MI (US)

(21) Appl. No.: 15/043,071

(22) Filed: Feb. 12, 2016

Related U.S. Application Data
(60) Provisional application No. 62/115,488, filed on Feb. 12, 2015.

(51) Int. Cl. C07K 16/28; A61K 47/48; A61K 39/395; A61K 45/06

(52) U.S. Cl. CPC C07K 16/2803; A61K 45/06; A61K 47/48561; A61K 47/48384; A61K 39/3955; C07K 2317/31; C07K 2317/732; C07K 2317/734; C07K 2317/41; A61K 2039/505

(57) ABSTRACT

Provided herein are compositions (e.g., killer cell immunoglobulin-like receptor (KIR)-targeting agents) that target a subset of T lymphocytes present in disease states (e.g., lupus and other autoimmune diseases) and methods of treating conditions and/or diseases therewith. In particular, anti-KIR antibodies, fragments thereof, or related compositions are provided for the treatment of conditions and/or diseases (e.g., lupus and other autoimmune diseases, atherosclerosis, etc.).

### CROSS-REFERENCE TO RELATED APPLICATION

The present invention claims priority to U.S. Provisional Patent Application Ser. No. 62/115,488 filed Feb. 12, 2015, which is incorporated herein by reference in its entirety.

### FIELD

Provided herein are compositions (e.g., killer cell immunoglobulin-like receptor (KIR)-targeting agents) that target a subset of T lymphocytes present in disease states (e.g., lupus and other autoimmune diseases) and methods of treating conditions and/or diseases therewith. In particular, anti-KIR antibodies, fragments thereof, or related compositions are provided for the treatment of conditions and/or diseases (e.g., lupus and other autoimmune diseases, atherosclerosis, etc.).

### BACKGROUND

T cells (T lymphocytes) from lupus patients have hypomethylated DNA and overexpress genes normally suppressed by DNA methylation that contribute to disease pathogenesis. We found that stimulatory and inhibitory killer cell immunoglobulin-like receptor (KIR) genes are aberrantly overexpressed on experimentally demethylated T cells. It has been suggested that aberrant T cell KIR expression may contribute to interferon (IFN) overproduction and macrophage killing in human lupus (Basu et al. J Immunol. 2009 Sep. 1; 183(5): 3481-3487; herein incorporated by reference in its entirety). Compositions and methods that target these T cells may be useful in the treatment of lupus.

### SUMMARY

Provided herein are compositions (e.g., killer cell immunoglobulin-like receptor (KIR)-targeting agents) that target a subset of T lymphocytes present in disease states (e.g., lupus and other autoimmune diseases) and methods of treating conditions and/or diseases therewith. In particular, anti-KIR antibodies, fragments thereof, or related compositions are provided for the treatment of conditions and/or diseases (e.g., lupus and other autoimmune diseases, atherosclerosis, etc.).

Experiments conducted during development of embodiments herein identified a subset of T lymphocytes that are CD3⁺CD4⁺CD28⁺PFN⁺CD11a^{hi}CD70⁺CD40L^{hi}KIR⁺ T lymphocytes. As used herein, this subset may be described as the “active lupus subset.” Because PFN is not a surface receptor, the subset may be referred to herein without referencing PFN status (e.g., “CD3⁺CD4⁺CD28⁺CD11a^{hi}CD70⁺CD40L^{hi}KIR⁺ T lymphocytes”). Similarly, since T lymphocytes are necessarily CD3⁺ the subset may be referred to herein without CD3 status (e.g., “CD4⁺CD28⁺PFN⁺CD11a^{hi}CD70⁺CD40L^{hi}KIR⁺ T lymphocytes”).

In some embodiments, provided herein are compositions comprising an antibody or antibody fragment that specifically binds an epitope presented by a killer cell immunoglobulin-like receptor (KIR) present on CD4⁺CD28⁺KIR⁺PFN⁺CD70⁺CD11a^{hi} CD40L^{hi} T lymphocytes from a subject with active lupus. In some embodiments, the antibody or antibody fragment binds one or more KIR proteins selected from the group consisting of KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. In some embodiments, the antibody or antibody fragment binds only one of KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. In some embodiments, the epitope is presented on the surface of the CD4⁺CD28⁺KIR⁺PFN⁺CD70⁺CD11a^{hi} CD40L^{hi} T lymphocytes. In some embodiments, the epitope is presented on the surface of at least 75% (e.g., 75%, 80%, 85%, 90%, 95%, 98%, 99%, 99.9%, and any ranges therein) of the CD4⁺CD28⁺KIR⁺PFN⁺CD70⁺CD11a^{hi}CD40L^{hi} T lymphocytes in a subject with active lupus. In some embodiments, the antibody or antibody fragment binds to epitopes on 20% or fewer (e.g., 0.1%, 0.2%, 0.5%, 1%, 2%, 5%, 10%, 15%, 20%, and any ranges therein) of natural killer (NK) cells. In some embodiments, the epitope is presented on the surface of 20% or fewer (e.g., 0.1%, 0.2%, 0.5%, 1%, 2%, 5%, 10%, 15%, 20%, and any ranges therein) of the NK cells in a subject with active lupus. In some embodiments, the antibody or antibody fragment is humanized, monoclonal, glycoengineered, a conjugate with a molecular agent (e.g., small molecule drug, toxin, peptide, polypeptide, antibody, etc.) configured to kill a cell to which the antibody or antibody fragment binds, bispecific, and/or multivalent. In some embodiments, the antibody or antibody fragment is bispecific and binds two KIR proteins selected from the group consisting of KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1.

In some embodiments, provided herein are methods of treating a disease or condition (e.g., autoimmune disease (e.g., lupus, rheumatoid arthritis, Sjogren's syndrome, progressive systemic sclerosis (PSS), multiple sclerosis, etc.) comprising administering to a subject an antibody or antibody fragment that specifically binds an epitope presented by a killer cell immunoglobulin-like receptor (KIR) present on CD4⁺CD28⁺KIR⁺PFN⁺CD70⁺CD11a^{hi}CD40L^{hi} T lymphocytes in the subject, wherein the epitope is present on 20% or fewer (e.g., 0.1%, 0.2%, 0.5%, 1%, 2%, 5%, 10%, 15%, 20%, and any ranges therein) of natural killer (NK) cells in the subject. In some embodiments, the epitope is presented by at least 75% (e.g., 75%, 80%, 85%, 90%, 95%, 98%, 99%, 99.9%, and any ranges therein) of CD4⁺CD28⁺KIR⁺PFN⁺CD70⁺CD11a^{hi} CD40L^{hi} T lymphocytes in the subject. In some embodiments, the antibody or antibody fragment binds one or more KIR protein selected from the group consisting of KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. In some embodiments, the antibody or antibody fragment binds only one of KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. In some embodiments, the epitope is presented on the surface of the CD4⁺CD28⁺KIR⁺PFN⁺CD70⁺CD11a^{hi} CD40L^{hi} T lymphocytes. In some embodiments, the antibody or antibody fragment is humanized, monoclonal, glycoengineered, a conjugate with a molecular agent (e.g., small molecule drug, toxin, peptide, polypeptide, antibody, etc.) configured to kill a cell to which the antibody or antibody fragment binds, bispecific, and/or multivalent. In some embodiments, the antibody or antibody fragment is bispecific and binds two KIR proteins selected from the group consisting of KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. In some embodiments, the antibody or antibody fragment is coadministered with one or more additional treatments for the disease or condition (e.g., autoimmune disease). In some embodiments, the disease or condition is an autoimmune disease and the additional treatments are selected from immunosuppressives and anti-inflammatories. In some embodiments, the autoimmune disease is systemic lupus erythematosus (SLE). In some embodiments, the antibody or antibody fragment is coadministered with one or more additional treatments for lupus.

In some embodiments, provided herein are methods of treating an autoimmune disease comprising administering to a subject an agent that selectively targets CD3⁺CD4⁺CD28⁺CD11a^{hi}CD70⁺CD40L^{hi}KIR⁺ T lymphocytes in the subject. In some embodiments, the agent comprises an antibody or antibody fragment. In some embodiments, the agent binds an epitope presented on the surface of the CD3⁺CD4⁺CD28⁺CD11a^{hi}CD70⁺CD40L^{hi}KIR⁺ T lymphocytes. In some embodiments, the epitope is presented on the surface of at least 75% of the CD3⁺CD4⁺CD28⁺CD11a^{hi}CD70⁺CD40L^{hi}KIR⁺ T lymphocytes in the subject or in subjects with active lupus. In some embodiments, the epitope is presented on the surface of less than 20% of NK cells in the subject or in subjects with active lupus. In some embodiments, the agent binds one or more KIR proteins present on the CD3⁺CD4⁺CD28⁺CD11a^{hi}CD70⁺CD40L^{hi}KIR⁺ T lymphocytes. In some embodiments, the agent binds one or more KIR protein selected from the group consisting of KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. In some embodiments, the agent binds only one of KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. In some embodiments, the agent is multispecific, recognizing two or more epitopes presented on the CD3⁺CD4⁺CD28⁺CD11a^{hi}CD70⁺CD40L^{hi}KIR⁺ T lymphocytes. In some embodiments, the multispecific agent binds at least one KIR protein selected from the group consisting of KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. In some embodiments, the multispecific agent binds at least two KIR proteins selected from the group consisting of KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. In some embodiments, the agent is a conjugate with a molecular agent configured to kill a cell to which the agent binds. In some embodiments, the molecular agent is selected from the selected from the group consisting of small molecule drugs, toxins, peptides, polypeptides, and antibodies. In some embodiments, the agent is coadministered with one or more additional treatments for the autoimmune disease. In some embodiments, the additional treatments are selected from immunosuppressives and anti-inflammatories. In some embodiments, the autoimmune disease is selected from the group consisting of systemic lupus erythematosus (SLE), rheumatoid arthritis, Sjogren's syndrome, progressive systemic sclerosis (PSS), multiple sclerosis. In some embodiments, the autoimmune disease is SLE.

In some embodiments, provided herein are methods of treating atheroscleoris in a subject comprising administering to the subject an agent that selectively targets CD3⁺CD4⁺CD11a^{hi}CD70⁺CD40L^{hi}KIR⁺ T lymphocytes in the subject. In some embodiments, the CD3⁺CD4⁺CD11a^{hi}CD70⁺CD40L^{hi}KIR⁺ T lymphocytes are CD28⁺. In some embodiments, the subject suffers from an autoimmune disease selected from the group consisting of systemic lupus erythematosus (SLE), rheumatoid arthritis, Sjogren's syndrome, progressive systemic sclerosis (PSSmultiple sclerosis. In some embodiments, the subject does not suffer from an autoimmune disease selected from the group consisting of systemic lupus erythematosus (SLE), rheumatoid arthritis, Sjogren's syndrome, progressive systemic sclerosis (PSS), multiple sclerosis. In some embodiments, the agent binds to CD3⁺CD4⁺CD28⁺CD11a^{hi}CD70⁺CD40L^{hi}KIR⁺ T lymphocytes CD3⁺CD4⁺CD28⁻CD11a^{hi}CD70⁺CD40L^{hi}KIR⁺ T lymphocytes. In some embodiments, the agent comprises an antibody or antibody fragment. In some embodiments, the agent binds an epitope presented on the surface of the CD3⁺CD4⁺CD11a^{hi}CD70⁺CD40L^{hi}KIR⁺ T lymphocytes. In some embodiments, the agent binds one or more KIR proteins present on the CD3⁺CD4⁺CD11a^{hi}CD70⁺CD40L^{hi}KIR⁺ T lymphocytes. In some embodiments, the agent binds one or more KIR protein selected from the group consisting of KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. In some embodiments, the agent binds only one of KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. In some embodiments, the agent is multispecific, recognizing two or more epitopes presented on the CD3⁺CD4⁺CD11a^{hi}CD70⁺CD40L^{hi}KIR⁺ T lymphocytes. In some embodiments, the multispecific agent binds at least one KIR protein selected from the group consisting of KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. In some embodiments, the agent binds at least two KIR proteins selected from the group consisting of KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. In some embodiments, the agent is a conjugate with a molecular agent configured to kill a cell to which the agent binds. In some embodiments, the molecular agent is selected from the selected from the group consisting of small molecule drugs, toxins, peptides, polypeptides, and antibodies. In some embodiments, the agent is coadministered with one or more additional treatments for atherosclerosis (e.g., cholesterol and/or blood pressure lowering medications).

### BRIEF DESCRIPTION OF THE DRAWINGS

FIGS. 1A-D. Patients with active lupus have a CD4⁺CD28⁺KIR⁺CD11a^{hi} T cell subset. PBMCs from a healthy donor (A), a lupus patient with mildly active disease (B), and an active lupus patient (C) were stained with fluorochorome-conjugated antibodies to CD3, CD4, CD28, CD11a, and KIR then analyzed gating on the CD3⁺CD4⁺CD28⁺ subset. (D) KIR⁺CD11a^{high} T cell subset size in 12 lupus patients showing a correlation between the subset size and SLEDAI (P=0.002).

FIGS. 2A-B. (A) Murine splenocytes were stimulated with PHA, treated or not with 5-azaC, then KirL1 mRNA was measured in CD4⁺ T cells relative to GAPDH by RT-PCR. (B) Double transgenic mice were given Dox or not in their drinking water, then KirL1 mRNA measured in CD4+ splenocytes as in panel A.

FIGS. 3A-B.) Double transgenic mice were given Dox (B) or not (A) in their drinking water, then KirL1 expression was measured on CD4+ splenocytes by flow cytometry.

FIG. 4. CD40L on CD4⁺ T cells. MS; methyl supplemented. MR; methyl restricted.

FIG. 5. In vivo depletion of CD4⁺CD11a^{hi}CD40L^{hi} T cells by anti-KirL1.

FIG. 6. Anti-KirL1 hybridoma secretes Ab reactive with demethylated T cells.

FIG. 7. Correlation between Disease activity and the CD4+CD28+CD11a^{hi} Kir+ subset in Rheumatic Diseases.

FIG. 8. CD4+CD11a^{high} CD28+Kir+ T cells from Active SLE co-express CD40L and CD70.

FIGS. 9A-E. Kir+ —Subset on CD4 T Cells from a Very Active (>6 SLEDAI) SLE, Female peripheral blood mononuclear cells (PBMC) were stained with Pacific Blue-CD3, FITC-CD70; FITC-PFN, PE-Kir (cocktail), APC-CD11a, PE-Cy5-CD28, APC-Cy7-CD4, and Biotin-CD40L/PECy7Avidin.

FIG. 10. Exemplary flow cytometric analysis strategy. PBMC from patients with systemic autoimmune rheumatic diseases or PHA stimulated, 5-azaC treated PBMC from healthy subjects were stained with fluorochrome conjugated antibodies to CD3, CD4, CD28, CD11a, CD70, CD40L and the KIR gene family, then analyzed by gating on CD3⁺CD4⁺CD28⁺ cells and comparing the levels of KIR and CD11a then CD40L and CD70 on the cells.

FIGS. 11A-D. 5-Azacytidine treatment of PHA-stimulated PBMC induces the expression of KIR proteins on a subset of CD4⁺CD28⁺CD11a^{hi} T cells. PBMC were stimulated for 18-24 hrs with PHA followed by treatment with 2.5 μM 5-azacytidine or saline for 72 hrs. The cells were then stained with fluorochrome-conjugated antibodies to methylation-sensitive gene products, fixed and analyzed by flow cytometry as described in Methods. Gating was established to select CD3⁺CD4⁺CD28⁺ T cells. The CD3⁺CD4⁺CD28⁺ T cells were then analyzed for CD11a and KIR using a cocktail of 5 PE-conjugated anti-KIR antibodies and imaged on a FACS Synergy flow cytometer. (A), Untreated T cells. (B), 5-azaC treated T cells. (C), 5-azaC treated PBMC stained only with the cocktail of anti-KIR antibodies. (D), 5-azaC treated T cells stained with antibodies to CD3, CD4, CD28, CD11a, CD70 and CD40L but not KIR. The figure shown is representative of 9 separate cultures.

FIGS. 12A-I. CD3⁺CD4⁺CD28⁺CD11a^{hi}KIR⁺ T cells in patients with rheumatic autoimmune diseases. The selected examples are representative of 4-7 patients for each level of disease activity. (A), Inactive SLE (SLEDI 0-2); (B), mildly active lupus (SLEDAI 4-5); (C), active lupus (SLEDAI 6 or more); (D), inactive RA; (E), active RA; (F) inactive systemic sclerosis; (G), active systemic sclerosis. Idiopathic retroperitoneal fibrosis served as a control. Disease activity (H-I) was clinically determined as described in the Methods. Gating was established based on single antibody and full minus one controls for each experiment. The rectangles indicate KIR⁺ cells selected in quadrant 2. Numbers above the rectangles are the percent of total cells.

FIGS. 13A-D. Relationship between disease activity and size of the CD3⁺CD4⁺CD28⁺CD11a^{hi}KIR⁺ T cell subset in patients with rheumatic diseases. (A-C), the size of the CD3⁺CD4⁺CD28⁺CD11a^{hi}KIR⁺ T cell subset in patients is plotted against their disease activity/involvement scores. P values shown were determined by linear regression. (D), Average+/−SEM of the size of the CD3⁺CD4⁺CD28⁺CD11a^{hi}KIR⁺ T cell subset from A-C. The numbers in the bars represent the number of subjects studied, and results are presented as the mean+SEM. P values shown were calculated using Student's t-test. Control, healthy subjects, retroperitoneal fibrosis (RPF), lupus (SLE) and SLEDAI scores 0-2, 4-6 and >6, inactive rheumatoid arthritis (RA(I)), active rheumatoid arthritis (RA(A), inactive progressive systemic sclerosis (SSc (I), active progressive systemic sclerosis (SSc (A)), and Sjogren's Syndrome.

FIGS. 14A-C. CD40L/CD70 co-expression on KIR⁺ versus KIR⁻ CD4⁺CD28⁺ T Cells. The dotted line demarks positive from negative CD40L staining.

FIGS. 15A-M. Exemplary gating strategy for selection of the Kir⁺ subset. PBMC were stimulated 18-24 hr with PHA followed by treatment with 5-azacytidine for an additional 72 hrs. Non-specific binding sites on the cells were blocked and the cells were stained with marker-specific antibodies. (A), gating FSC versus SSC, all cells; (B), single cells selected based on forward scatter; (C), scatter-normal cells from B gated based on side scatter; (D), CD3⁺CD4⁺ cells selected from C; (E), CD3⁺CD4⁺CD28⁺ cells from D; (F), CD3⁺CD4⁺CD28⁺ cells analyzed for CD11a, and KIR. (G-M), Single antibody staining performed on single cells (from panel C) to establish positive staining controls and compensation for each fluorophore. (G), PB-CD3; (H), APC-Cy7-CD4; (I), PE-Cy5-CD28; (J), APC-Cy7-CD4; (K), PE-KIR; (L), FITC-CD70; (M) PECy7-CD40L.

### DEFINITIONS

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition (e.g., an autoimmune disorder (e.g., lupus), etc.).

As used herein, the term “antibody” refers to a whole antibody molecule or a fragment thereof (e.g., fragments such as Fab, Fab′, and F(ab′)₂), it may be a polyclonal or monoclonal antibody, a chimeric antibody, a humanized antibody, a human antibody, etc.

A native antibody typically has a tetrameric structure. A tetramer typically comprises two identical pairs of polypeptide chains, each pair having one light chain (in certain embodiments, about 25 kDa) and one heavy chain (in certain embodiments, about 50-70 kDa). In a native antibody, a heavy chain comprises a variable region, V_{H}, and three constant regions, C_{H1}, C_{H2}, and C_{H3}. The V_{H} domain is at the amino-terminus of the heavy chain, and the C_{H3} domain is at the carboxy-terminus. In a native antibody, a light chain comprises a variable region, V_{L}, and a constant region, C_{L}. The variable region of the light chain is at the amino-terminus of the light chain. In a native antibody, the variable regions of each light/heavy chain pair typically form the antigen binding site. The constant regions are typically responsible for effector function.

In a native antibody, the variable regions typically exhibit the same general structure in which relatively conserved framework regions (FRs) are joined by three hypervariable regions, also called complementarity determining regions (CDRs). The CDRs from the two chains of each pair typically are aligned by the framework regions, which may enable binding to a specific epitope. From N-terminus to C-terminus, both light and heavy chain variable regions typically comprise the domains FR1, CDR1, FR2, CDR2, FR3, CDR3 and FR4. The CDRs on the heavy chain are referred to as H1, H2, and H3, while the CDRs on the light chain are referred to as L1, L2, and L3. Typically, CDR3 is the greatest source of molecular diversity within the antigen-binding site. H3, for example, in certain instances, can be as short as two amino acid residues or greater than 26. The assignment of amino acids to each domain is typically in accordance with the definitions of Kabat et al. (1991) Sequences of Proteins of Immunological Interest (National Institutes of Health, Publication No. 91-3242, vols. 1-3, Bethesda, Md.); Chothia, C., and Lesk, A. M. (1987) J. Mol. Biol. 196:901-917; or Chothia, C. et al. Nature 342:878-883 (1989). In the present application, the term “CDR” refers to a CDR from either the light or heavy chain, unless otherwise specified.

As used herein, the term “heavy chain” refers to a polypeptide comprising sufficient heavy chain variable region sequence to confer antigen specificity either alone or in combination with a light chain.

As used herein, the term “light chain” refers to a polypeptide comprising sufficient light chain variable region sequence to confer antigen specificity either alone or in combination with a heavy chain.

As used herein, when an antibody or other entity “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷M⁻¹ (e.g., >10⁷ M⁻¹, >10⁸ M⁻¹, >10⁹ M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

As used herein, the term “anti-KIR antibody” or “KIR antibody” refers to an antibody which specifically recognizes an antigen and/or epitope presented by one or more killer cell immunoglobulin-like receptors (KIRs). A “cross-reactive KIR antibody” refers to an antibody which specifically recognizes an antigen and/or epitope presented by more than one KIR. For example, a “KIR3D/KIR2D cross-reactive antibody” specifically recognizes an antigen and/or epitope presented by both KIR3D and KIR2D receptors.

As used herein, the term “monoclonal antibody” refers to an antibody which is a member of a substantially homogeneous population of antibodies that specifically bind to the same epitope. In certain embodiments, a monoclonal antibody is secreted by a hybridoma. In certain such embodiments, a hybridoma is produced according to certain methods known to those skilled in the art. See, e.g., Kohler and Milstein (1975) Nature 256: 495-499; herein incorporated by reference in its entirety. In certain embodiments, a monoclonal antibody is produced using recombinant DNA methods (see, e.g., U.S. Pat. No. 4,816,567). In certain embodiments, a monoclonal antibody refers to an antibody fragment isolated from a phage display library. See, e.g., Clackson et al. (1991) Nature 352: 624-628; and Marks et al. (1991) J. Mol. Biol. 222: 581-597; herein incorporated by reference in their entireties. The modifying word “monoclonal” indicates properties of antibodies obtained from a substantially-homogeneous population of antibodies, and does not limit a method of producing antibodies to a specific method. For various other monoclonal antibody production techniques, see, e.g., Harlow and Lane (1988) Antibodies: A Laboratory Manual (Cold Spring Harbor Laboratory, Cold Spring Harbor, N.Y.); herein incorporated by reference in its entirety.

As used herein, the term “antibody fragment” refers to a portion of a full-length antibody, including at least a portion antigen binding region or a variable region. Antibody fragments include, but are not limited to, Fab, Fab′, F(ab′)₂, Fv, scFv, Fd, diabodies, and other antibody fragments that retain at least a portion of the variable region of an intact antibody. See, e.g., Hudson et al. (2003) Nat. Med. 9:129-134; herein incorporated by reference in its entirety. In certain embodiments, antibody fragments are produced by enzymatic or chemical cleavage of intact antibodies (e.g., papain digestion and pepsin digestion of antibody) produced by recombinant DNA techniques, or chemical polypeptide synthesis.

For example, a “Fab” fragment comprises one light chain and the C_{H1} and variable region of one heavy chain. The heavy chain of a Fab molecule cannot form a disulfide bond with another heavy chain molecule. A “Fab”′ fragment comprises one light chain and one heavy chain that comprises additional constant region, extending between the C_{H1} and C_{H2} domains. An interchain disulfide bond can be formed between two heavy chains of a Fab′ fragment to form a “F(ab′)₂” molecule.

An “Fv” fragment comprises the variable regions from both the heavy and light chains, but lacks the constant regions. A single-chain Fv (scFv) fragment comprises heavy and light chain variable regions connected by a flexible linker to form a single polypeptide chain with an antigen-binding region. Exemplary single chain antibodies are discussed in detail in WO 88/01649 and U.S. Pat. Nos. 4,946,778 and 5,260,203; herein incorporated by reference in their entireties. In certain instances, a single variable region (e.g., a heavy chain variable region or a light chain variable region) may have the ability to recognize and bind antigen.

Other antibody fragments will be understood by skilled artisans.

As used herein, the term “chimeric antibody” refers to an antibody made up of components from at least two different sources. In certain embodiments, a chimeric antibody comprises a portion of an antibody derived from a first species fused to another molecule, e.g., a portion of an antibody derived from a second species. In certain such embodiments, a chimeric antibody comprises a portion of an antibody derived from a non-human animal fused to a portion of an antibody derived from a human. In certain such embodiments, a chimeric antibody comprises all or a portion of a variable region of an antibody derived from a non-human animal fused to a constant region of an antibody derived from a human.

A “humanized” antibody refers to a non-human antibody that has been modified so that it more closely matches (in amino acid sequence) a human antibody. A humanized antibody is thus a type of chimeric antibody. In certain embodiments, amino acid residues outside of the antigen binding residues of the variable region of the non-human antibody are modified. In certain embodiments, a humanized antibody is constructed by replacing all or a portion of a complementarity determining region (CDR) of a human antibody with all or a portion of a CDR from another antibody, such as a non-human antibody, having the desired antigen binding specificity. In certain embodiments, a humanized antibody comprises variable regions in which all or substantially all of the CDRs correspond to CDRs of a non-human antibody and all or substantially all of the framework regions (FRs) correspond to FRs of a human antibody. In certain such embodiments, a humanized antibody further comprises a constant region (Fc) of a human antibody.

The term “human antibody” refers to a monoclonal antibody that contains human antibody sequences and does not contain antibody sequences from a non-human animal. In certain embodiments, a human antibody may contain synthetic sequences not found in native antibodies. The term is not limited by the manner in which the antibodies are made. For example, in various embodiments, a human antibody may be made in a transgenic mouse, by phage display, by human B-lymphocytes, or by recombinant methods.

As used herein, the term “natural antibody” refers to an antibody in which the heavy and light chains of the antibody have been made and paired by the immune system of a multicellular organism. For example, the antibodies produced by the antibody-producing cells isolated from a first animal immunized with an antigen are natural antibodies. Natural antibodies contain naturally-paired heavy and light chains. The term “natural human antibody” refers to an antibody in which the heavy and light chains of the antibody have been made and paired by the immune system of a human subject.

Native human light chains are typically classified as kappa and lambda light chains. Native human heavy chains are typically classified as mu, delta, gamma, alpha, or epsilon, and define the antibody's isotype as IgM, IgD, IgG, IgA, and IgE, respectively. IgG has subclasses, including, but not limited to, IgG1, IgG2, IgG3, and IgG4. IgM has subclasses including, but not limited to, IgM1 and IgM2. IgA has subclasses including, but not limited to, IgA1 and IgA2. Within native human light and heavy chains, the variable and constant regions are typically joined by a “J” region of about 12 or more amino acids, with the heavy chain also including a “D” region of about 10 more amino acids. See, e.g., Fundamental Immunology (1989) Ch. 7 (Paul, W., ed., 2nd ed. Raven Press, N.Y.); herein incorporated by reference in its entirety.

The term “neutralizing antibody” or “antibody that neutralizes” refers to an antibody that reduces at least one activity of a polypeptide comprising the epitope to which the antibody specifically binds. In certain embodiments, a neutralizing antibody reduces an activity in vitro and/or In vivo. In some embodiments, by neutralizing the polypeptide comprising the epitope, the neutralizing antibody inhibits the capacity of the cell displaying the epitope. For example, a “KIR neutralizing antibody” reduces the capacity of one or more KIR proteins to bind ligand and/or transduce an activating or inhibitory signal.

As used herein, the term “glycoengineered”, as used herein, includes any manipulation of the glycosylation pattern of a naturally occurring or recombinant protein, polypeptide or a fragment thereof.

The term “antigen-binding site” refers to a portion of an antibody capable of specifically binding an antigen. In certain embodiments, an antigen-binding site is provided by one or more antibody variable regions.

The term “epitope” refers to any polypeptide determinant capable of specifically binding to an immunoglobulin or a T-cell or B-cell receptor. In certain embodiments, an epitope is a region of an antigen that is specifically bound by an antibody. In certain embodiments, an epitope may include chemically active surface groupings of molecules such as amino acids, sugar side chains, phosphoryl, or sulfonyl groups. In certain embodiments, an epitope may have specific three dimensional structural characteristics (e.g., a “conformational” epitope) and/or specific charge characteristics.

As used herein, the term “multivalent”, particularly when used in describing an agent that is an antibody, antibody fragment, or other binding agent, refers to the presence of two or more (e.g., 2, 3, 4, 5, 6, 7, 8, 9, 10, or more) antigen binding sites on the agent.

As used herein, the term “multispecific”, particularly when used in describing an agent that is an antibody, antibody fragment, or other binding agent, refers to the capacity to of the agent to bind two or more (e.g., 2, 3, 4, 5, 6, 7, 8, 9, 10, or more) targets (e.g., unrelated targets). For example, a bispecific antibody recognizes and binds to two different antigens.

An epitope is defined as “the same” as another epitope if a particular antibody specifically binds to both epitopes. In certain embodiments, polypeptides having different primary amino acid sequences may comprise epitopes that are the same. In certain embodiments, epitopes that are the same may have different primary amino acid sequences. Different antibodies are said to bind to the same epitope if they compete for specific binding to that epitope.

A “conservative” amino acid substitution refers to the substitution of an amino acid in a polypeptide with another amino acid having similar properties, such as size or charge. In certain embodiments, a polypeptide comprising a conservative amino acid substitution maintains at least one activity of the unsubstituted polypeptide. A conservative amino acid substitution may encompass non-naturally occurring amino acid residues, which are typically incorporated by chemical peptide synthesis rather than by synthesis in biological systems. These include, but are not limited to, peptidomimetics and other reversed or inverted forms of amino acid moieties. Naturally occurring residues may be divided into classes based on common side chain properties, for example: hydrophobic: norleucine, Met, Ala, Val, Leu, and Ile; neutral hydrophilic: Cys, Ser, Thr, Asn, and Gln; acidic: Asp and Glu; basic: His, Lys, and Arg; residues that influence chain orientation: Gly and Pro; and aromatic: Trp, Tyr, and Phe. Non-conservative substitutions may involve the exchange of a member of one of these classes for a member from another class; whereas conservative substitutions may involve the exchange of a member of one of these classes for another member of that same class.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families (see above). The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein, the term “selectively” (e.g., as in “selectively targets,” “selectively binds,” etc.) refers to the preferential association of an agent (e.g., antibody or antibody fragment) for a particular entity (e.g., antigen, antigen presenting cell, etc.). For example, an agent selectively targets a particular cell population if it preferentially associates (e.g., binds an epitope or set of epitopes presented thereon) with that cell population over another cell population (e.g., all other cell populations present in a sample). The preferential association may be by a factor of at least 2, 4, 6, 8, 10, 20, 50, 100, 10³, 10⁴, 10⁵, 10⁶, or more, or ranges there between. An agent that X-fold selectively targets a particular cell populations, associates with that cell population by at least X-fold more than other cell populations present.

The term “effective dose” or “effective amount” refers to an amount of an agent, e.g., an antibody, that results in the reduction of symptoms in a patient or results in a desired biological outcome. In certain embodiments, an effective dose or effective amount is sufficient to treat or reduce symptoms of a disease or condition (e.g., an autoimmune disorder (e.g., lupus), etc.).

As used herein, the terms “administration” and “administering” refer to the act of giving a drug, prodrug, or other agent, or therapeutic to a subject or In vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

The term “treatment” encompasses both therapeutic and prophylactic/preventative measures unless otherwise indicated. Those in need of treatment include, but are not limited to, individuals already having a particular condition (e.g., lupus) as well as individuals who are at risk of acquiring a particular condition or disorder (e.g., those having a genetic or epigenetic predisposition, etc.). The term “treating” refers to administering an agent to a subject for therapeutic and/or prophylactic/preventative purposes.

A “therapeutic agent” refers to an agent that may be administered In vivo to bring about a therapeutic and/or prophylactic/preventative effect.

A “therapeutic antibody” refers to an antibody that may be administered In vivo to bring about a therapeutic and/or prophylactic/preventative effect.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term pharmaceutical composition” refers to the combination of an active agent (e.g., binding agent) with a carrier, inert or active, making the composition especially suitable for diagnostic or therapeutic use in vitro, In vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

### DETAILED DESCRIPTION

Provided herein are compositions (e.g., killer cell immunoglobulin-like receptor (KIR)-targeting agents) that target a subset of T lymphocytes present in disease states (e.g., lupus and other autoimmune diseases) and methods of treating conditions and/or diseases therewith. In particular, anti-KIR antibodies, fragments thereof, or related compositions are provided for the treatment of conditions and/or diseases (e.g., lupus and other autoimmune diseases, atherosclerosis, etc.).

DNA methylation patterns are established during differentiation, and serve to silence genes that are inappropriate for the function of any given cell type. The methylation patterns are then replicated each time a cell divides by DNA methyltransferase 1 (Dnmt1). Inhibiting Dnmt1 in dividing cells prevents methylation of the newly synthesized DNA strand and activates expression of those genes silenced by DNA methylation, but for which the cell expresses the necessary transcription factors (Richardson, B. 2007. Nat Clin Pract Rheumatol 3:521-527; herein incorporated by reference in its entirety).

It has been experimentally demonstrated that cloned or polyclonal antigen specific CD4+ T cells treated with the Dnmt inhibitor 5-azacytidine (5-azaC) lose restriction to nominal antigen and kill autologous or syngeneic macrophages without added antigen in a class II MHC restricted fashion (Richardson et al. 1992. Arthritis Rheum 35:647-662; herein incorporated by reference in its entirety). The autoreactivity was traced to LFA-1 (CD11a/CD18) overexpression, caused by demethylation of ITGAL (CD11a) regulatory regions, and causing LFA-1 overexpression by transfection caused a similar MHC-specific autoreactivity and macrophage killing (Richardson, et al. 1994. Arthritis Rheum 37:1363-1372; herein incorporated by reference in its entirety). The autoreactivity may be due to overstabilization of the normally low affinity interaction between the TCR and self-class II MHC molecules without the appropriate antigenic peptides in the antigen binding cleft, caused by the additional LFA-1 molecules surrounding the immunologic synapse (Richardson, et al. 1994. Arthritis Rheum 37:1363-1372; herein incorporated by reference in its entirety). These class II MHC-responsive T cells resemble the semi-allogeneic CD4+ T cells that cause a lupus-like disease in the chronic graft-vs-host disease mouse model (Rolink & Gleichmann. 1983. J Exp Med 158:546-55; herein incorporated by reference in its entirety). It was subsequently reported that CD4+ T cells made autoreactive either by treatment with 5-azaC, or by LFA-1 transfection, are sufficient to cause a lupus-like disease when injected into syngeneic mice (Yung et al. 1996. J Clin Invest 97:2866-2871; herein incorporated by reference in its entirety).

Further studies demonstrated that patients with active but not inactive lupus have hypomethylated T cell DNA (Richardson et al. 1990. Arthritis Rheum 33:1665-1673; herein incorporated by reference in its entirety) and a CD4+ T cell subset that overexpresses LFA-1 (Richardson et al. 1992. Arthritis Rheum 35:647-662; herein incorporated by reference in its entirety), due to demethylation of the same ITGAL regulatory regions affected by 5-azaC (Lu, et al. 2002. Arthritis Rheum 46:1282-1291; herein incorporated by reference in its entirety). The LFA-1^{high} T cell subset, isolated by flow cytometry from patients with active lupus, selectively induced apoptosis in autologous but not allogeneic macrophages in a class II MHC specific fashion, similar to 5-azaC treated LFA-t^{high} human and mouse CD4+ T cells (Richardson et al. 1992. Arthritis Rheum 35:647-662; herein incorporated by reference in its entirety). It was also found that patients with active but not inactive lupus have apoptotic monocytes circulating in their peripheral blood, suggesting that the killing occurs In vivo (Richardson et al. 1996. Arthritis Rheum 39:1432-1434; herein incorporated by reference in its entirety). Since apoptotic chromatin is sufficient to induce lupus-like autoimmunity with anti-DNA antibodies in mice, and macrophages clear apoptotic debris (Walport. 2000. Nat Genet 25:135-136; herein incorporated by reference in its entirety), it is contemplated that macrophage killing contributes to the development of anti-DNA antibodies and lupus-like autoimmunity in the adoptive transfer model as well as in lupus patients, by both increasing release of apoptotic material and impairing its clearance. Inducing macrophage apoptosis in mice with clodronate-containing vesicles causes apoptosis of the macrophages which engulf them. Mice receiving clodronate-containing but not control vesicles developed anti-DNA antibodies, and the vesicles accelerated lupus onset in lupus-prone mice (Denny et al. 2006. J Immunol 176:2095-2104; herein incorporated by reference in its entirety). This accumulation of necrotic and late apoptotic cells causes release of nuclear material that when combined with SLE IgG, stimulates plasmacytoid dendritic cells to secrete IFNα (Lovgren et al. 2004. Arthritis Rheum 50:1861-1872; herein incorporated by reference in its entirety), a cytokine that plays a critical role in lupus flares (Niewold. 2011. J Interferon Cytokine Res 31:887-892; herein incorporated by reference in its entirety).

Studies have traced the human lupus T cell DNA methylation defect to low Dnmt1 levels, caused by decreased ERK pathway signaling, and treating human CD4+ T cells with ERK pathway (MEK) inhibitors decreased Dnmt1 levels and caused LFA-1 overexpression and class II WIC-specific autoreactivity (Gorelik & Richardson. 2009. Autoimmun Rev 8:196-198; herein incorporated by reference in its entirety). Further, treating mouse CD4+ T cells with MEK inhibitors then injecting them into syngeneic mice caused a lupus-like disease (Deng et al. 2003. Arthritis Rheum 48:746-756; herein incorporated by reference in its entirety), demonstrating pathogenicity. The importance of decreased T cell ERK pathway signaling was confirmed by creating a double transgenic mouse strain in which expression of a dominant negative MEK is selectively induced in T cells by adding doxycycline (dox) to their drinking water. Giving dox decreases T cell Dnmt1, demethylates T cell DNA, causes overexpression of methylation-sensitive genes on T cells, and induces anti-dsDNA antibodies and an “interferon signature” in double transgenic C57BL6 mice, which lack lupus genes (Sawalha et al. 2008. Genes Immun 9:368-378), and anti-dsDNA and an immune complex glomerulonephritis in double transgenic lupus-prone C57BL6×SJL mice (Strickland et al. 2012. J Autoimmun 38:J135-143.1; herein incorporated by reference in its entirety).

The human lupus T cell ERK pathway defect was traced to PKCδ (Gorelik et al. 2007. J Immunol 179:5553-5563; herein incorporated by reference in its entirety), an upstream regulator of MEK and ERK which is inactivated in lupus T cells by oxidative damage (Gorelik et al. 2007. J Immunol 179:5553-5563; herein incorporated by reference in its entirety). PKCδ “knockout” mice develop lupus (Mecklenbrauker et al. 2002. Nature 416:860-865; herein incorporated by reference in its entirety), suggesting that T cell PKCδ inactivation by reactive oxygen species may contribute to lupus-like autoimmunity. In experiments conducted during development of embodiments described herein, a double transgenic mouse strain has been developed in which a dominant negative PKCδ rather than the dnMEK is selectively induced in T cells by dox. These mice also develop a lupus-like disease with anti-dsDNA antibodies (p=0.010 by ANOVA, dox vs. control) and proteinuria (43±5.7 vs. 2.5±2.5 mg/dl, dox vs. control, p=0.03). Together these reports indicate a mechanism by which environmental agents associated with lupus, such as UV light, infections, silica exposure and smoking, all of which cause oxidative stress (Patel & Richardson. 2013. Arthritis Res Ther 15:201; herein incorporated by reference in its entirety), trigger lupus flares through inhibitory effects on T cell ERK pathway signaling and DNA methylation. It has also been reported that the anti-oxidant N-acetylcysteine decreases flare severity in lupus patients (Lai et al. 2012. Arthritis Rheum 64:2937-2946; herein incorporated by reference in its entirety).

Additional T cell genes activated by DNA demethylation were sought using 5-azaC and Affymetrix arrays, and evidence for their demethylation and overexpression was tested in CD4+ T cells from patients with active lupus. These studies identified the cytotoxic molecule perforin (Kaplan et al. 2004. J Immunol 172:3652-3661; Lu et al. 2003. J Immunol 170:5124-5132; herein incorporated by reference in their entireties), and the B cell costimulatory molecules CD70 (Lu et al. 2005. J Immunol 174:6212-6219; Oelke et al. 2004. Arthritis Rheum 50:1850-1860; herein incorporated by reference in their entireties) and CD40L (Lu et al. 2007. J Immunol 179:6352-6358; herein incorporated by reference in its entirety), as genes demethylated and overexpressed in both 5-azaC treated T cells and T cells from patients with active lupus, suggesting that these molecules might be therapeutic targets. However, perforin is intracellular, preventing antibody-based approaches, and CD70 was overexpressed on T cells from patients with inactive as well as active lupus, suggesting that CD70 overexpression may not play a prominent role in lupus flares. Others reported that CD40L can accelerate lupus in mice (Higuchi et al. 2002. J Immunol 168:9-12; herein incorporated by reference in its entirety), and that antibodies to CD40L treat lupus in mice (Early et al. 1996. J Immunol 157:3159-3164; herein incorporated by reference in its entirety), making CD40L a potentially attractive target. However, therapeutic trials in human lupus patients were complicated by thromboses, possibly because human platelets express CD40L (Sidiropoulos & Boumpas. 2004. Lupus 13:391-397; herein incorporated by reference in its entirety). CD40L is on the X chromosome, and one X chromosome is inactivated in women by mechanisms that include DNA methylation, so CD4+ T cells from women have one unmethylated, expressed CD40L gene and one methylated, silenced gene, while CD4+ T cells from men have only one, unmethylated CD40L gene. It was found that CD40L is demethylated and overexpressed in 5-azaC treated female CD4+ T cells and in CD4+ T cells from women with active lupus, while 5-azaC and lupus disease activity had no significant effect on expression of the single demethylated CD40L gene in T cells from men (Lu, et al. 2007. J Immunol 179:6352-6358; herein incorporated by reference in its entirety).

Array studies also revealed that 5-azaC activates expression of the killer-immunoglobulin-like receptor gene family in CD4+ T cells. KIR proteins are normally expressed by NK cells, recognize MHC-class I molecules with locus and allele specificity, and regulate cytotoxic and IFNγ responses (Vilches & Parham. 2002. Annu Rev Immunol 20:217-251; herein incorporated by reference in its entirety). The human KIR gene locus encodes up to 14 highly homologous proteins that are clonally expressed on the surface of NK cells, with the non-expressed genes silenced only by DNA methylation, and inhibiting DNA methylation in NK cells is sufficient to activate expression of multiple KIR genes (Chan et al. 2003. J Exp Med 197:245-255; herein incorporated by reference in its entirety). Normal T cells do not express KIR genes, even when stimulated with mitogen. However, it was found that treating CD4+ T cells from healthy subjects with 5-azaC similarly induces expression of multiple KIR genes by both demethylating the KIR promoters as well as through effects on transcription factors (Liu et al. 2009. Clin Immunol 130:213-224; herein incorporated by reference in its entirety). It was also found that the KIR gene family is demethylated and expressed on CD4+ T cells from patients with active but not inactive lupus, and that the number of CD4+KIR+ T cells is directly proportional to the SLEDAI (Basu et al. 2009. J Immunol 183:3481-3487; herein incorporated by reference in its entirety). Antibodies to the stimulatory molecule KIR2DL4 triggered IFNγ release by human lupus T cells, and IFNγ production was directly proportional to disease activity. Similarly, cross-linking the inhibitory molecule KIR3DL1 prevented the autoreactive macrophage killing that characterizes human lupus T cells (Basu et al. 2009. J Immunol 183:3481-3487; herein incorporated by reference in its entirety).

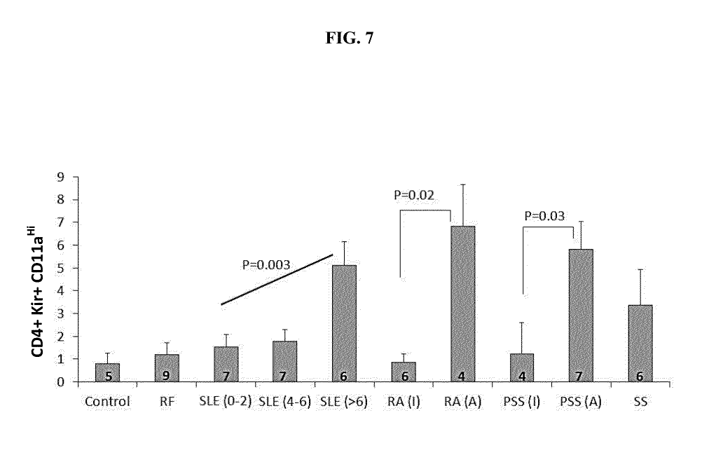

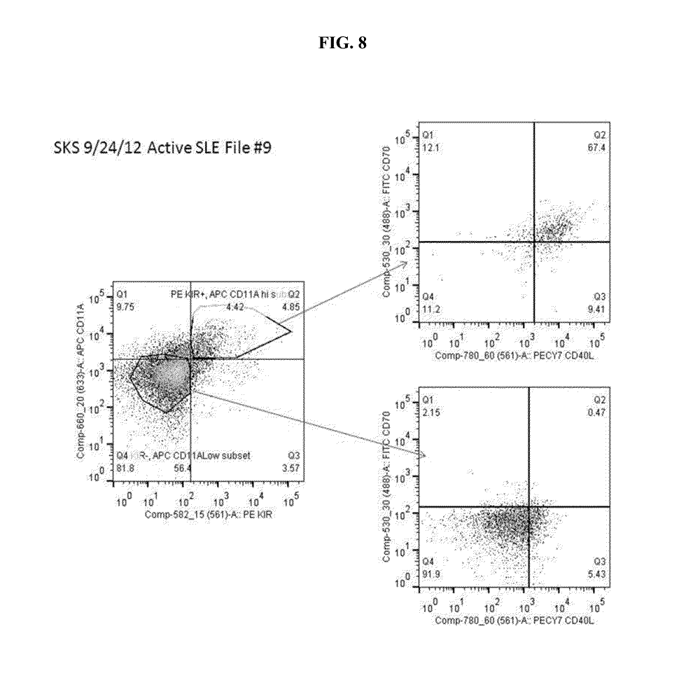

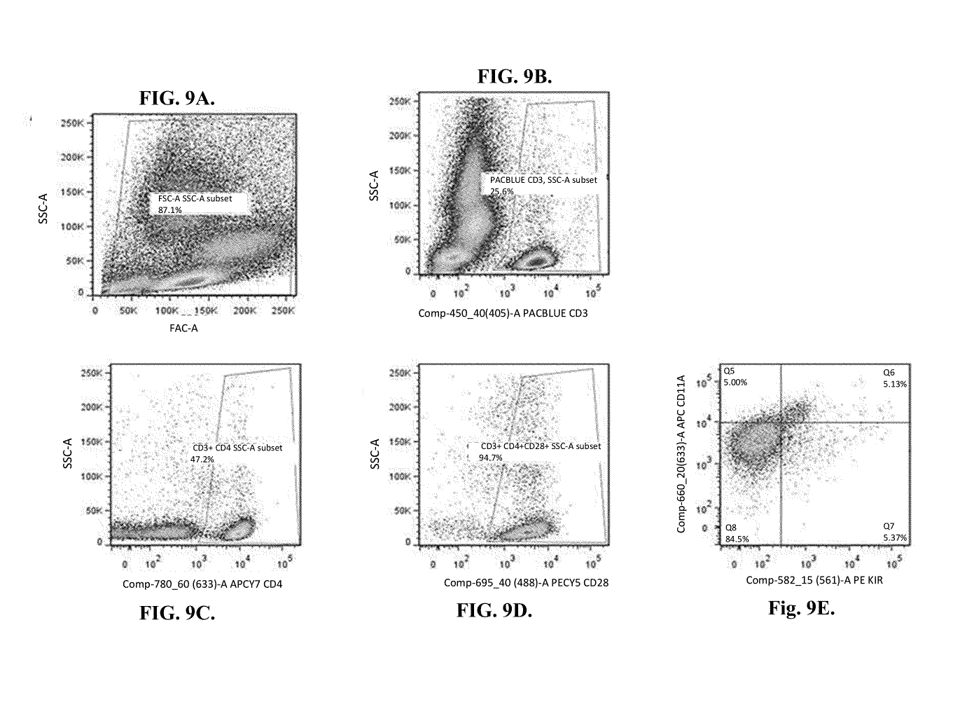

Experiments conducted during development of embodiments described herein demonstrated that disregulation of DNA methylation causes the overexpression of multiple immune molecules on CD4+ T cells, rendering them autoreactive. These CD4⁺ T cells overexpressing CD11a, CD40L and KIR proteins kill autologous macrophages and stimulate antibody production by B cells in vitro, and induce lupus-like autoimmunity in murine models. Polychromatic flow cytometry was used to immunophenotype freshly isolated CD4⁺ T cell subsets from donors with diverse rheumatic diseases including SLE, rheumatoid arthritis (RA), Sjogren's syndrome, progressive systemic sclerosis (PSS) and idiopathic retroperitoneal fibrosis. A subset of CD4⁺CD28⁺ demethylated T cells co-expressed the lupus-associated CD11a^{hi}, CD40L, KIR and CD70 surface molecules and intracellular perforin (PFN) by eight color flow cytometry. The size of this subset correlated with disease activity in SLE, RA and PSS (See FIG. 7) and was observed in mitogen-activated experimentally demethylated T cells, but not in T cells treated with mitogen alone. Experiments indicate the involvement of this T cell subset in causing tissue destruction in autoimmunity (e.g., lupus) and provides a target for therapeutic intervention with anti-KIR compositions (e.g., antibodies).

Experiments conducted during development of embodiments herein, using multicolor flow cytometry to compare CD11a, CD70, CD40L and KIR expression on CD3+CD4+CD28+ T cells to their expression on experimentally-demethylated CD3⁺CD4⁺CD28⁺ T cells and CD3⁺CD4⁺CD28⁺ T cells from patients with active lupus and other autoimmune diseases, demonstrated that patients with active autoimmune rheumatic diseases have a previously undescribed CD3⁺CD4⁺CD28⁺CD11a^{hi}CD70⁺CD40L^{hi}KIR⁺ T cell subset. Data indicate that subset plays an important role in flares of lupus and related autoimmune rheumatic diseases, providing a biomarker for disease activity and a therapeutic target for treatment of lupus flares.

Experiments conducted during development of embodiments herein indicate that the CD3⁺CD4⁺CD28⁺CD11a^{hi}CD70⁺CD40L^{hi}KIR⁺ T cell subset senesces into a CD3⁺CD4⁺CD28⁻CD11a^{hi}CD70⁺CD40L^{hi}KIR⁺ T cell subset which results in the formation and rupture of atherosclerotic plaques in subjects suffering from lupus or other autoimmune diseases as well as subjects without autoimmune disease. In some embodiments, treatment of subjects with agents targeting the CD3⁺CD4⁺CD28⁺CD11a^{hi}CD70⁺CD40L^{hi}KIR⁺ T cell subset prevents formation of the CD3⁺CD4⁺CD28⁻CD11a^{hi}CD70⁺CD40L^{hi}KIR⁺ T cell subset and subsequent formation and rupture of atherosclerotic plaques.

KIR genes (e.g., KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1) are clonally expressed on natural killer (NK) cells. As a result of this clonal expression, each NK cell expresses only a subset of the KIR receptors. Normal CD4⁺ T cells do not express KIR genes. Experiments conducted during development of embodiments described herein demonstrate that patients with active lupus exhibit a T cell subset that is CD4⁺CD28⁺KIR⁺PFN⁺CD70⁺CD11a^{hi} CD40L^{hi} (referred to herein as the “active lupus subset”), and that multiple KIR genes (e.g., the entire KIR gene family) are expressed in T cells of the active lupus subset. In some embodiments, compositions (e.g., antibodies) are provided that target one or more KIR proteins on CD4⁺ T cells from patients with active lupus and target only a subset (e.g., <20%, <15%, <10%, 9%, <8%, <7%, <6%, <5%, <4%, <3, <2%, <1%, <0.5%, <0.1%) of NK cells. In some embodiments, targeting of T cells with anti-KIR compositions (e.g., antibodies) results in destruction, killing, prevention of proliferation, etc. of such T cells. In some embodiments, the therapeutic strategies described herein are not limited to lupus, but find utility in the treatment and/or prevention of other diseases, conditions, or disease states (e.g., active disease) in which aberrant KIR expression is causative and/or observed (e.g., autoimmune disorders (e.g., rheumatoid arthritis, Sjogren's syndrome, progressive systemic sclerosis (PSS), etc.), atherosclerotic plaque growth and rupture, etc.).

Since normal T cells do not express KIR genes, in some embodiments, KIR proteins serve as a biomarker for demethylated T cells, T cells in lupus patients, etc. Further, since multiple KIR genes are expressed by demethylated T cells but are expressed clonally on NK cells, in some embodiments, antibodies targeting a single KIR protein deplete a majority of demethylated T cells, but only a subset of NK cells. In some embodiments, targeting inhibitory KIR proteins with non-cytotoxic antibodies suppresses the demethylated T cells but only a subset of NK cells.

Experiments have demonstrated that a humanized anti-KIR antibody that increases NK killing is well tolerated and is being used to treat acute myelogenous leukemia (AML) in people (Romagne et al. 2009. Blood 114:2667-2677; herein incorporated by reference in its entirety).

Experiments were conducted during development of embodiments described herein demonstrating the population of CD4⁺CD28⁺KIR⁺PFN⁺CD70⁺CD11a^{hi} CD40L^{hi} T cells in subjects suffering from active SLE. Experiments demonstrate that the cells of this population express and/or display their surface a plurality (e.g., all) of KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. NK cells comprise a diverse population of cells, which each only display a small subset of the KIR proteins. In some embodiments, provided herein are compositions and methods for targeting (e.g., killing) CD4⁺CD28⁺KIR⁺PFN⁺CD70⁺CD11a^{hi} CD40L^{hi} T cells with an antibody to one or more KIR proteins. In some embodiments, while the majority (e.g., all) of the CD4⁺CD28⁺KIR⁺PFN⁺CD70⁺CD11a^{hi}CD40L^{hi} T cells are targeted by such methods and compositions, only a small subset of NK cells are (e.g., because most NK cells do not display the particular KIR protein targeted).

In some embodiments, provided herein are multispecific antibodies or antibody fragments with two or more antigen binding sites which are specific for different epitopes. In some embodiments, the different epitopes are on different proteins (e.g., two different KIR proteins, one KIR protein and another cell surface receptor presented on the active lupus subset). In some embodiments, such multispecific antibodies or fragments are readily produced by recombinant expression of nucleic acid encoding the polypeptide chains of the antibody. In one embodiment, the multivalent antibody comprises a dimerization domain and multiple (e.g., three or more) antigen binding sites. In some embodiments, the antibody or antibody fragment is specific for the active lupus subset due to multiple antigen binding sites for different cell surface proteins displayed on the active lupus subset.

In some embodiments, a pharmaceutical composition comprising the antibodies disclosed herein includes an acceptable carrier and is formulated into a suitable dosage form according to administration modes. Pharmaceutical preparations suitable for administration modes are known, and generally include surfactants that facilitate transport across the membrane. Such surfactants may be derived from steroids, or may be cationic lipids such as N-[1-(2,3-dioleyloxy)propyl]-N,N,N-trimethylammonium chloride (DOTMA), or various compounds such as cholesterol hemisuccinate and phosphatidyl glycerol.

For oral administration, the pharmaceutical composition may be presented as discrete units, for example, capsules or tablets; powders or granules; solutions, syrups or suspensions (edible foam or whip formulations in aqueous or non-aqueous liquids); or emulsions.

For parenteral administration, the pharmaceutical composition may include aqueous and non-aqueous sterile injection solutions which may contain anti-oxidants, buffers, bacteriostats and solutes which render the formulation substantially isotonic with the blood of the intended recipient; and aqueous and non-aqueous sterile suspensions which may include suspending agents and thickening agents. Excipients available for use in injectable solutions include, for example, water, alcohol, polyols, glycerin, and vegetable oils. Such a composition may be presented in unit-dose (single dose) or multiple dose (several doses) containers, for example, sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

The pharmaceutical composition may include antiseptics, solubilizers, stabilizers, wetting agents, emulsifiers, sweeteners, colorants, odorants, salts, buffering agents, coating agents, or anti-oxidants.

Compositions may comprise, in addition to the antibody or antibodies described herein, a therapeutically active agent (e.g., drug), additional antibodies, etc.

The present composition may be formulated into dosage forms for use in humans or veterinary use. Composition may be administered to humans or non-human animals such as non-human primates, rodents, canines, felines, bovines, equines, porcines, etc. Antibodies may be administered alone or in combination with another treatment.

The antibody composition may be administered in a pharmaceutically effective amount in a single- or multiple-dose. The pharmaceutical composition may be administered via any of the common routes, as long as it is able to reach the desired tissue. Thus, the present composition may be administered via oral or parenteral (e.g., subcutaneous, intramuscular, intravenous, or intradermal administration) routes, and may be formulated into various dosage forms. In one embodiment, the formulation is an injectable preparation. Intravenous, subcutaneous, intradermal, intramuscular and dropping injectable preparations are possible.

Antibodies may be coupled to a drug for delivery to a treatment site or coupled to a detectable label to facilitate imaging of a site comprising cells of interest, such as CD4⁺CD28⁺KIR⁺PFN⁺CD70⁺CD11a^{hi} CD40L^{hi} T lymphocytes. Methods for coupling antibodies to drugs and detectable labels are well known in the art, as are methods for imaging using detectable labels. Labeled antibodies may be employed in a wide variety of assays, employing a wide variety of labels. Detection of the formation of an antibody-antigen complex between an antibody of the invention and an epitope of interest can be facilitated by attaching a detectable substance to the antibody. Suitable detection means include the use of labels such as radionuclides, enzymes, coenzymes, fluorescers, chemiluminescers, chromogens, enzyme substrates or co-factors, enzyme inhibitors, prosthetic group complexes, free radicals, particles, dyes, and the like. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material is luminol; examples of bioluminescent materials include luciferase, luciferin, and aequorin; and examples of suitable radioactive material include ¹²⁵I, ¹³¹I, ³⁵S, or ³H. Such labeled reagents may be used in a variety of well-known assays, such as radioimmunoassays, enzyme immunoassays, e.g., ELISA, fluorescent immunoassays, and the like.

An antibody may be conjugated to another therapeutic moiety. Such antibody conjugates can be used for modifying a given biological response; the drug moiety is not to be construed as limited to classical chemical therapeutic agents. For example, the drug moiety may be a protein or polypeptide possessing a desired biological activity. Techniques for conjugating such therapeutic moiety to antibodies are well known. See, for example, Arnon et al. (1985) “Monoclonal Antibodies for Immunotargeting of Drugs in Cancer Therapy,” in Monoclonal Antibodies and Cancer Therapy, ed. Reisfeld et al. (Alan R. Liss, Inc.), pp. 243-256; ed. Hellstrom et al. (1987) “Antibodies for Drug Delivery,” in Controlled Drug Delivery, ed. Robinson et al. (2d ed; Marcel Dekker, Inc.), pp. 623-653; Thorpe (1985) “Antibody Carriers of Cytotoxic Agents in Cancer Therapy: A Review,” in Monoclonal Antibodies '84: Biological and Clinical Applications, ed. Pinchera et al. pp. 475-506 (Editrice Kurds, Milano, Italy, 1985); “Analysis, Results, and Future Prospective of the Therapeutic Use of Radiolabeled Antibody in Cancer Therapy,” in Monoclonal Antibodies for Cancer Detection and Therapy, ed. Baldwin et al. (Academic Press, New York, 1985), pp. 303-316; and Thorpe et al. (1982) Immunol. Rev. 62:119-158; herein incorporated by reference in their entireties.

Alternatively, an antibody, or antibody fragment thereof, can be conjugated to a second antibody, or antibody fragment thereof, to form an antibody heteroconjugate as described in U.S. Pat. No. 4,676,980; herein incorporated by reference in its entirety. In addition, linkers may be used between the labels and the antibodies of the invention (e.g. U.S. Pat. No. 4,831,175; herein incorporated by reference in its entirety). In some embodiments, antibodies or antibody fragments with three (e.g., trivalent) or more (tetravalent, multivalent, etc.) functional antigen binding sites are provided (See, e.g., WO 2001077342; herein incorporated by reference in its entirety).

Antibodies of the invention may also be attached to a solid support. Additionally, antibodies of the invention, or functional antibody fragments thereof, can be chemically modified by covalent conjugation to a polymer to, for example, increase their circulating half-life. In some embodiments the polymers may be selected from polyoxyethylated polyols and polyethylene glycol (PEG). PEG is soluble in water at room temperature and has the general formula: R(O—CH₂—CH₂)—O—R where R can be hydrogen, or a protective group such as an alkyl or alkanol group, and where n is 2-2000.

Water-soluble polyoxyethylated polyols may also be employed. They include polyoxyethylated sorbitol, polyoxyethylated glucose, polyoxyethylated glycerol (POG), and the like. Another drug delivery system that can be used for increasing circulatory half-life is the liposome.

Antibodies may be provided in purified form. Typically, the antibody will be present in a composition that is substantially free of other polypeptides e.g. where less than 90% (by weight), usually less than 60% and more usually less than 50% of the composition is made up of other polypeptides.

Antibodies of the invention can be of any isotype (e.g. IgA, IgG, IgM (e.g., an alpha, gamma or mu heavy chain). Within the IgG isotype, antibodies may be IgG1, IgG2, IgG3 or IgG4 subclass. Antibodies may have a kappa or a lambda light chain.

In some embodiments, antibodies described herein are coadministered with an additional therapeutic for the disease or condition being treated. For example, when treating lupus (e.g., active lupus), antibodies may be coadministered with one or more of: nonsteroidal anti-inflammatory drugs (e.g., naproxen sodium, ibuprofen, etc.), antimalarial drugs (e.g., hydroxychloroquine), corticosteroids (e.g., prednisone, etc.), immunosuppressants (e.g., azathioprine, mycophenolate, leflunomide, methotrexate, etc.), etc.

### EXPERIMENTAL

### Example 1

### Identification of the Demethylated KIR+CD4+CD28+ T Cell Subset

Decreased Dnmt1 levels cause generalized T cell DNA demethylation (Richardson, B. 2007. Primer: epigenetics of autoimmunity. Nat Clin Pract Rheumatol 3:521-527; herein incorporated by reference in its entirety). Experiments were conducted during development of embodiments described herein to demonstrate that T cell DNA demethylation results in demethylation and co-overexpression of KIR and the other genes normally suppressed by DNA methylation in the same cells. Another CD4+ T cell subset, that is characterized by loss of CD28 (CD4+CD28−) also has low Dnmt1 levels and co-overexpresses KIR and the other genes normally silenced only by DNA methylation (Chen et al. 2009. J Leukoc Biol; Li et al. 2010; Liu et al. 2009. Clin Immunol 132:257-265; herein incorporated by reference in their entireties). However, CD4+CD28− T cells are “senescent” with short telomeres, decreased replicative potential and apoptosis resistance (Dumitriu et al. 2009. Cardiovasc Res 81:11-19; Weng. 2009. Trends Immunol 30:306-312; herein incorporated by reference in their entireties). In contrast to the KIR+CD4+CD28+ T cells associated with lupus, KIR+CD4+CD28− T cells arise with aging and in chronic inflammatory diseases including rheumatoid arthritis (RA) and others (Nakajima, et al. 2002. Circulation 105:570-575; Dumitriu et al. 2009. Cardiovasc Res 81:11-19; herein incorporated by reference in their entireties) as well as in patients with lupus (Basu et al. 2009. J Immunol 183:3481-3487; herein incorporated by reference in its entirety). CD4+CD28− T cells infiltrate atherosclerotic plaques and are implicated in plaque rupture and myocardial infarction (Dumitriu et al. 2009. Cardiovasc Res 81:11-19; herein incorporated by reference in its entirety). In some embodiments, compositions and/or methods for inhibiting or depleting the CD4+CD28-KIR+ subset and/or CD4+CD28+KIR+ subset, are provided.

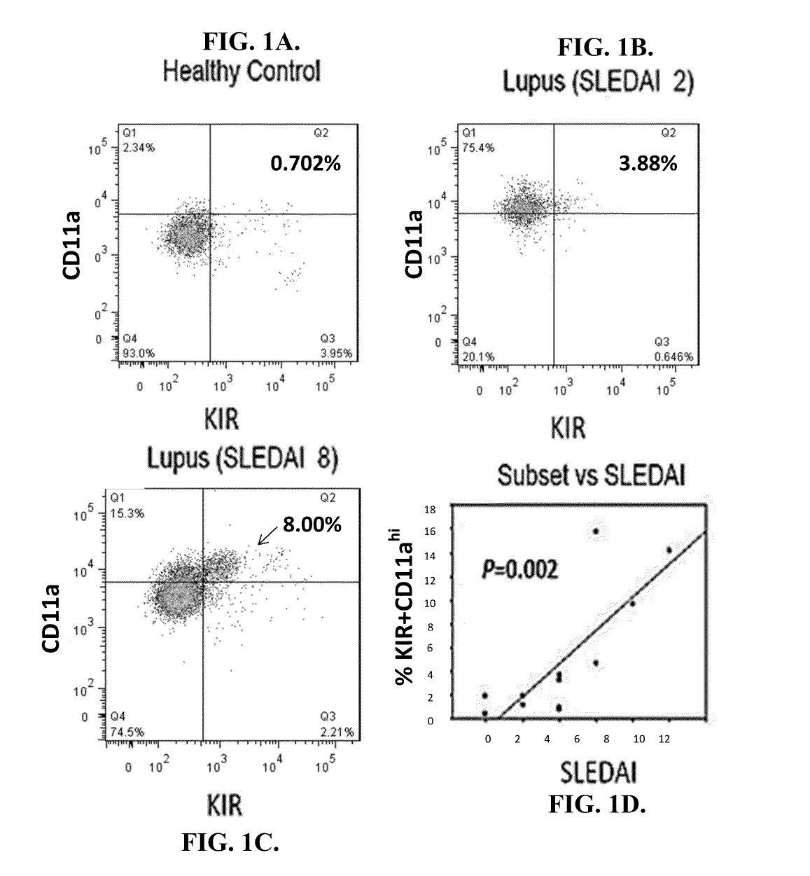

Co-overexpression of LFA-1 and CD40L, both implicated in lupus pathogenesis (Yung, et al. 1996. J Clin Invest 97:2866-2871), on KIR+CD4+CD28+ T cells was tested in 12 female lupus patients with a range of SLEDAI scores, using multicolor flow cytometry. FIG. 1 shows PBMC from: (A) a healthy control, and (B) a woman with mildly active lupus (SLEDAI 2), stained with anti-CD3-Pacific blue, anti-CD4-APC-Cy7, anti-CD28-PERCP, anti-CD11a-APC, PECy7-avidin-biotin-anti-CD40L and a cocktail of PE-conjugated anti-KIR antibodies, then analyzed by flow cytometry, gating on CD3+CD4+CD28+ cells. Panel B shows the CD4+CD28+KIR+CD40L^{hi}CD11a^{hi} subset (arrow) in the patient with mildly active lupus. The subset is not seen in the healthy control (panel A). Panel C shows the CD4+CD28+KIR+CD40L^{hi}CD11a^{hi} subset (arrow) in a patient with more active disease (SLEDAI=8) and the subset is larger. Panel D shows a graph of subset size vs. SLEDAI in 12 lupus patients. The subset size increases with disease activity (p=0.002 by regression).

Since the mechanism causing autoimmunity in the dox inducible T cell dnMEK transgenic mouse model (Sawalha et al. 2008. Genes Immun 9:368-378; Strickland et al. 2012. J. Autoimmun 38:J135-143; herein incorporated by reference in their entireties) closely resembles the ERK pathway defect in human lupus T cells (Deng et al. 2001. Arthritis Rheum 44:397-407; herein incorporated by reference in its entirety), experiments were conducted to determine whether dox caused development of a similar, demethylated KIR+ subset in mice. Human and mouse NK cells use structurally divergent families of receptors to recognize classical MHC class I molecules. MHC class I recognition by mouse NK cells is mediated by the Klr (Ly49) family of the C-type lectin-like receptors (Takei et al. 1997. Immunol Rev 155:67-77; herein incorporated by reference in its entirety). However, mice have recently been found to have three KIR genes, KIRL1, KIRL2 and KIR3DL1, with KIR3DL1 likely being an allele of KIRL1 (Wilson et al. 2007. Immunogenetics 59:641-651; herein incorporated by reference in its entirety). The mouse KIR genes are structurally homologous to the human KIR genes, and are expressed on NK1.1⁺ T cells but not on mature CD4+ and CD8+ T cells (Welch et al. 2003. Immunogenetics 54:782-790; herein incorporated by reference in its entirety). In mice KIRL1 is expressed on a subset of NK1.1+CD3+ lymphocytes that are CD1d-independent, but not on NK1.1+CD3− NK cells or conventional CD1d-restricted NKT cells.

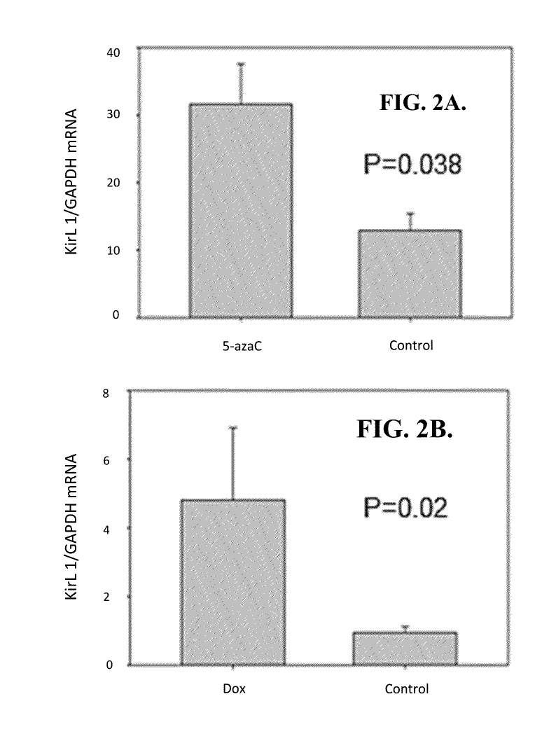

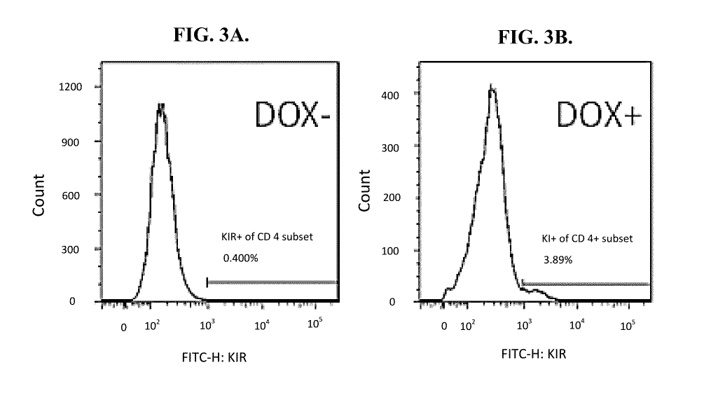

To test if the mouse T cell KIR genes are also activated by DNA methylation inhibition, murine splenocytes were stimulated with ConA, treated with 5-azaC, and KIRL1 mRNA measured relative to GAPDH by RT-PCR in CD4+ T cells bead purified by negative selection to remove NK and other non-CD4+ T cells. The demethylated cells overexpressed KIRL1 mRNA (n=4, p=0.038) (FIG. 2A). Similar to the 5-azaC treated cells, CD4+ T cells from tet-on C57BL6×SJL dnMEK mice receiving dox also had higher KIRL1 mRNA levels than controls without dox (n=14, p=0.02) (FIG. 2B). Flow cytometry, using a rat anti-mouse KIRL1 monoclonal antibody (6G10) demonstrated a 5.7-fold increase in CD4⁺KIR⁺NK1.1⁻ cells in transgenic mice receiving dox and a 3.1-fold increase in CD4⁺KIR⁺NK1.1⁻ cells in T cells treated with 5-azaC, indicating that KIR was induced on T cells and not NK cells. FIGS. 3A-B shows KIR expression in the tet-on mouse with higher KIR expression (3.9% of total CD4+ T cells), similar in size to the subset found in human lupus patients (FIG. 1). These results indicate that mice with decreased T cell Dnmt1 levels, caused by ERK pathway signaling abnormalities that resemble those in human lupus, also develop a KIR⁺ T cell subset as well as lupus-like autoimmunity (Sawalha et al. 2008. Genes Immun 9:368-378; herein incorporated by reference in its entirety).

Experiments were conducted during development of embodiments described herein to determine whether dox induces expression of other genes normally suppressed by DNA methylation on the KIR+ cells. SJL mice were given dox for 6 weeks, sacrificed, then splenocytes stained with Pacific Blue-CD3; FITC-CD8; PE-CD40L; PE-Cy5-CD4; Avidin-APC-Cy7/biotin-KIRL1; PERCP-Cy5.5-CD11a. Mice receiving dox developed a CD4⁺KIRL1⁺CD11a^{hi}CD40L⁺ subset that resembles the human lupus subset shown in FIG. 1, and represented 1.09% of the total CD11a+ cells, while controls had only 0.007% CD4⁺KIRL1⁺CD11a^{hi}CD40L⁺ cells.

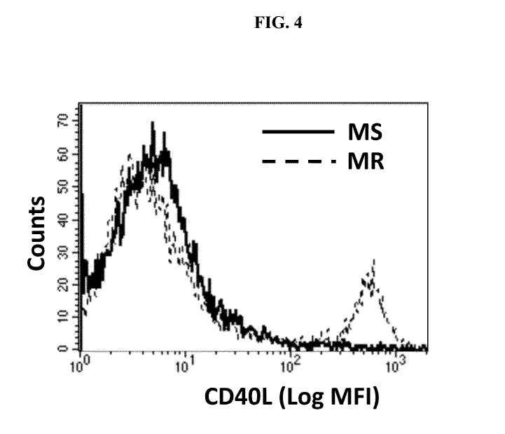

Further evidence demonstrating that the demethylated T cell subset is responsible for the autoimmunity was provided using a dnMEK model in which we induced lupus-like disease with dox in their drinking water and then fed diets enriched or low in methyl donors (Strickland et al. 2013. Arthritis Rheum 65:1872-1881; herein incorporated by reference in its entirety). Female mice receiving the methyl donor restricted (MR) diet and dox demethylated the T cell CD40LG promoter, developed a CD4+CD40L^{hi} subset (FIG. 4) and had high titer anti-dsDNA antibodies with severe kidney disease (2.9±1.6 vs 6.9±1.5% CD40L+, control vs MR, mean±SEM, p=0.036). In contrast female mice receiving the methyl donor supplemented (MS) diet and dox did not demethylate the CD40LG promoter or overexpress CD40L (3.0+0.5 vs 2.9±1.6% CD40L+), and had lower titer anti-dsDNA antibodies as well as significantly less kidney disease (Strickland et al. 2013. Arthritis Rheum 65:1872-1881; herein incorporated by reference in its entirety).

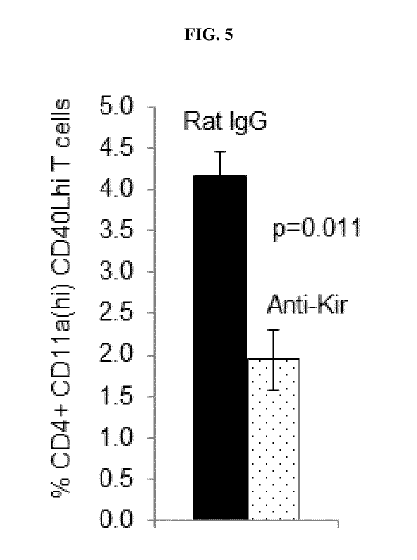

Experiments were conducted during development of embodiments described herein to determine if rat anti-KIR depleted KIR+CD4+ T cells in the dnMEK model. Adult (8-12 week old) double transgenic female mice were given dox for 6 weeks then 0.5 mg purified rat IgG or purified rat anti-KIRL1 monoclonal antibody was injected i.v. via the tail vein. Two days later the mice were given a second i.v. injection with the rat IgG or anti-KIRL1 antibody, then 24 hours later the mice were sacrificed and splenocytes isolated. Non-specific Fc-receptor binding sites were blocked with 10% horse serum/PBS/0.001% azide then the cells were washed, stained with PE-CD40L, PE-Cy5-CD4, FITC-CD11a, and analyzed by flow cytometry (FIG. 5). The CD4⁺CD11a^{high}CD40L⁺ subset represented 4.16±0.30% (mean+SEM) of total CD4+ T cells in the mice receiving control rat IgG, but only 1.94±0.36% of the mice receiving anti-KIR (p=0.011, n=6). These results thus support feasibility of depleting the LFA-1 overexpressing subset using anti-KIR. The mice tolerated the antibody injections well, with no evidence for a cytokine storm.

### Example 2

### Cytotoxic Mouse Anti-MR Antibodies

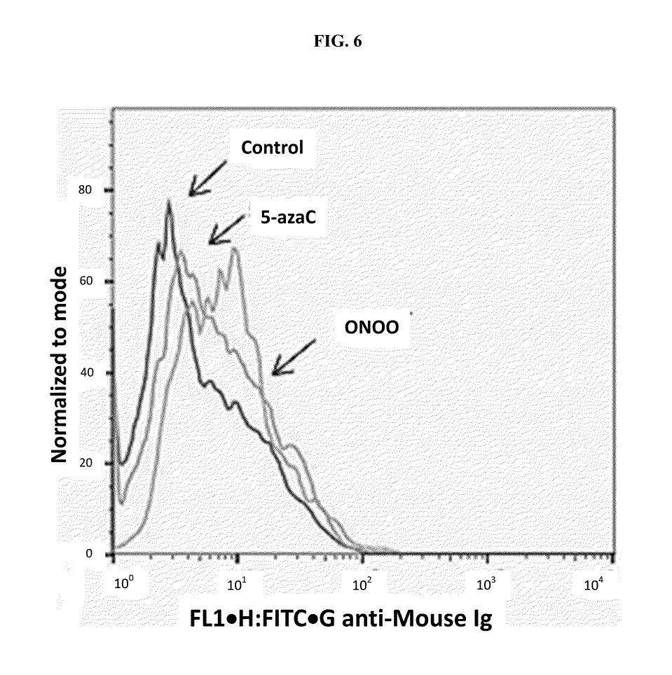

Since the rat-anti-mouse KIR antibodies used in experiments described in Example 1 stimulated production of neutralizing mouse-anti-rat antibodies, mouse anti-mouse KIR mAbs were generated. Mice were immunized with a KLH conjugated KIRL1 peptide representing the exterior domain (amino acids 164-178). Hybridomas were produced and screened by ELISA using the KIRL1 peptide and peptide conjugated to BSA to avoid KLH reactive antibodies and to obtain antibodies reactive with both linear and conformational determinants. Positive supernatants were confirmed by flow cytometry. FIG. 6 shows the staining of ConA-stimulated untreated murine CD4+ T cells and ConA-stimulated CD4+ T cells treated with the DNA methylation inhibitors 5-azaC or ONOO⁻ (Li et al. 2014. Arthritis Rheumatol 66:1574-1582; herein incorporated by reference in its entirety) with the supernatant from hybridoma 2A6 and detected using a FITC-conjugated goat anti-mouse Ig secondary antibody specific for mouse Ig. The hybridoma supernatant recognizes determinants on demethylated but not control CD4+ T cells (FIG. 6) as well as the KIR+KY NKT cell line.

KIR-positive wells are further assessed by flow cytometry using KIR+ murine KY cells. Ig isotype is determined by ELISA and flow cytometry, and IgG2a anti-KIR antibodies are selected for further testing since they participate in both complement dependent as well as antibody dependent cell mediated cytotoxicity (Lux & Nimmerjahn. 2013. J Clin Immunol 33 Suppl 1:S4-8; White et al. 2013. Cancer Immunol Immunother 62:941-948; Liu et al. 2008. Immunol Rev 222:9-27; herein incorporated by reference in their entireties). Positive hybridomas are subjected to three rounds of subcloning, and specificity confirmed using Western blot. Positive clones are assessed for complement dependent cytotoxic activity against mouse KY cells in vitro using complement dependent cytotoxicity protocols (Kohler et al. 1977. J Immunol 119:1979-1986; herein incorporated by reference in its entirety), and for the ability to deplete NK1.1(+) T cells in mice using flow cytometry.

Evidence that the antibody detects the same CD4+KIR+LFA-1^{hi}CD40L^{hi} subset as 6G10 (FIGS. 3A-B), is tested using splenocytes from tet-on dnMEK mice with and without dox in their drinking water and multicolor flow cytometry, staining with Pacific Blue-CD3, FITC-CD8, PE-CD40L, APC-Cy7-CD4, Avidin-APC/biotin-KIRL1, and PERCP-Cy5.5-CD11a.

Confirmation is obtained that the antibody detects the same CD4+KIR+LFA-1^{hi}CD40L^{hi} subset as 6G10, followed by experiments testing depletion of this subset by antibodies in vitro as detected by multicolor flow cytometry, using splenocytes from the tet-on dnMEK mice with and without dox and complement dependent cytotoxicity assays as described above.

As an alternate approach, the extracellular domain of KIRL1 is expressed in insect (SF9) cells—to produce a His-tagged KIRL1 protein that assumes a conformation and glycosylation closer to that found on mammalian cells and is used to generate monoclonal antibodies in mice.

### Example 3

### Treatment of Lupus with Anti-MR Antibodies

Experiments are conducted to identify depleting the KIR+ T cell subset with the anti-KIR Ab as a preventative of the development of lupus in dox-inducible dnMEK murine model on a C57BL6×SJL background. As noted above, the KIR gene family provides a therapeutic target, since KIR genes are not expressed on normal human CD4+CD28+ T cells, but are expressed on the demethylated T cell subset. Although human KIR genes are expressed clonally on NK cells, these cells are functionally impaired in lupus (Stohl et al. 1996. Arthritis Rheum 39:1840-1851; Ohtsuka et al. 1998. J Immunol 160:2539-2545; herein incorporated by reference in their entireties). Further, the entire KIR gene family is expressed by demethylated human T cells but clonally on normal NK cells (Chan et al. 2003. J Exp Med 197:245-255; herein incorporated by reference in its entirety), so targeting single KIR genes is contemplated to deplete demethylated CD4+ T cells but only a subset of NK cells. KIR genes are also expressed on “senescent” CD4+CD28− T cells (Liu et al. 2009. Clin Immunol 132:257-265; herein incorporated by reference in its entirety), and lupus patients develop this subset, as do people with other chronic inflammatory diseases as well as with aging. However, this subset is implicated in atherosclerotic plaque development and rupture (Dumitriu et al. 2009. Cardiovasc Res 81:11-19; herein incorporated by reference in its entirety), implicating anti-KIR antibodies in not only the treatment of human lupus, but also preventing the cardiovascular complications associated with SLE as well as with other diseases associated with atherosclerosis.

Anti-KIR antibodies are purified from the supernatants of the cultured hybridoma line by purification over protein G columns. The antibody is dialyzed against PBS, protein concentration determined using the bicinchoninic acid method, then filter sterilized (Shreedhar et al. 1998. J Immunol 160:3783-3789; herein incorporated by reference in its entirety). Isotype matched control antibodies are purchased from commercial sources such as Thermo Scientific Pierce Antibodies. Female mice are given dox or glucose in the drinking water as before, and injected i.p. with 100 μg anti-KIR or control IgG weekly. Urine protein is measured by dipstick and anti-dsDNA antibodies measured by ELISA every 4 weeks as described by our group using 5-10 mice/group (Strickland et al. 2013. Diet influences expression of autoimmune associated genes and disease severity by epigenetic mechanisms in a transgenic lupus model. Arthritis Rheum; herein incorporated by reference in its entirety). Maximal anti-dsDNA antibody levels develop by week 12. At each time point five mice from each group are sacrificed and depletion of the CD4+KIR+CD11a^{hi}CD40L^{hi} subset determined by flow cytometric analysis of splenocytes as in FIG. 5 and confirmed by RT-PCR. The kidneys are studied for histologic evidence of glomerulonephritis by routine hematoxylin and eosin staining as well as by immunofluorescence to detect IgG deposition. Both are scored on a 0-4+ scale and analyzed. Evidence for inflammatory lung and liver disease, seen in the adoptive transfer models is also sought. Controls include mice not receiving doxycycline.

The ability of the anti-KIR antibodies to deplete the CD4+KIR+CD11a^{hi}CD40L^{hi} subset and prevent the development of anti-DNA antibodies and kidney disease is compared to mice receiving control IgG using a two sample t-test with sufficient sample size. Otherwise, the Wilcoxon test or Fisher's exact test is used.

### Example 4

### Expression of Methylation-Sensitive Genes in 5-azaC Treated Cells

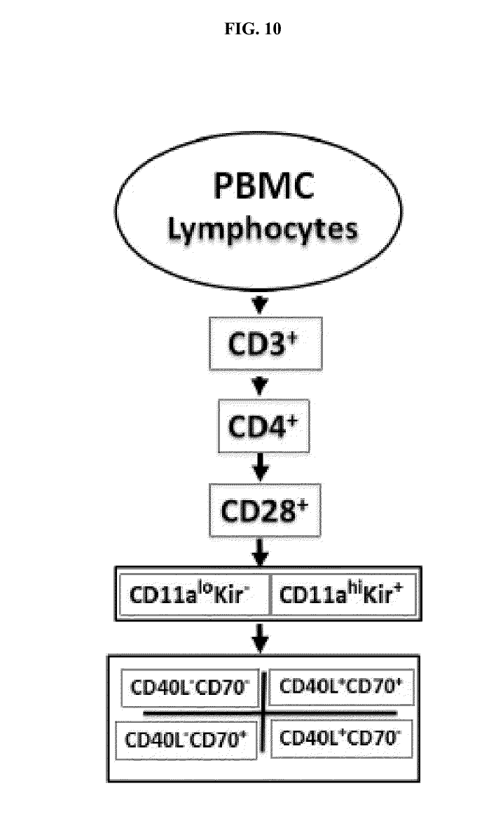

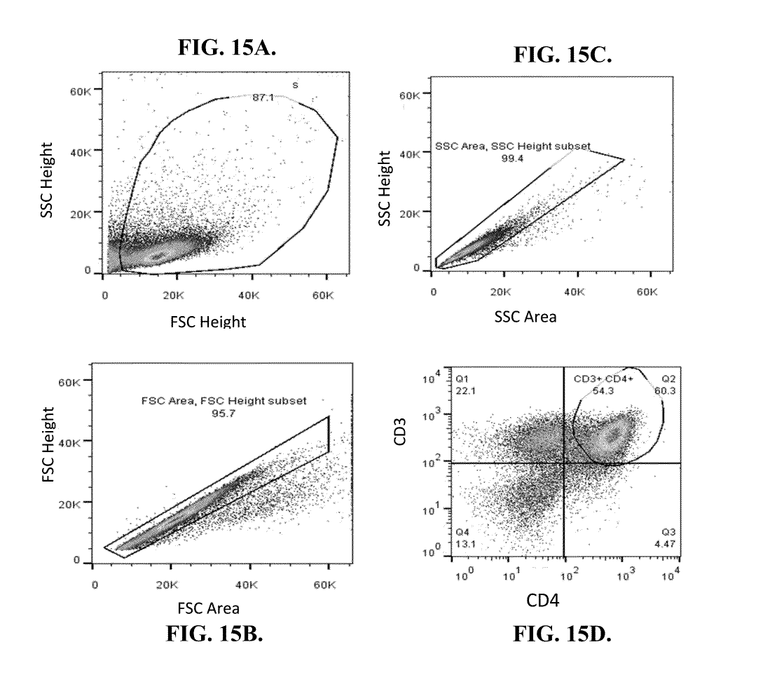

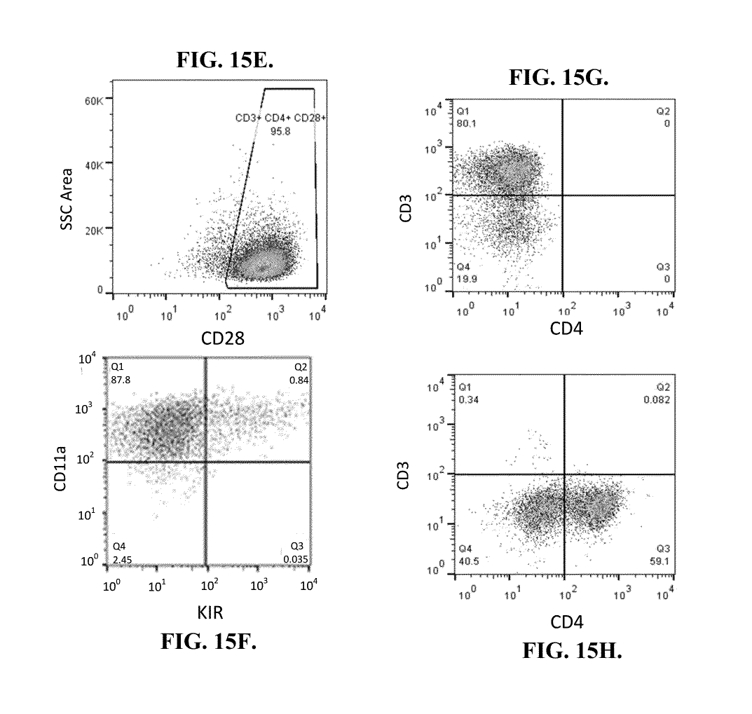

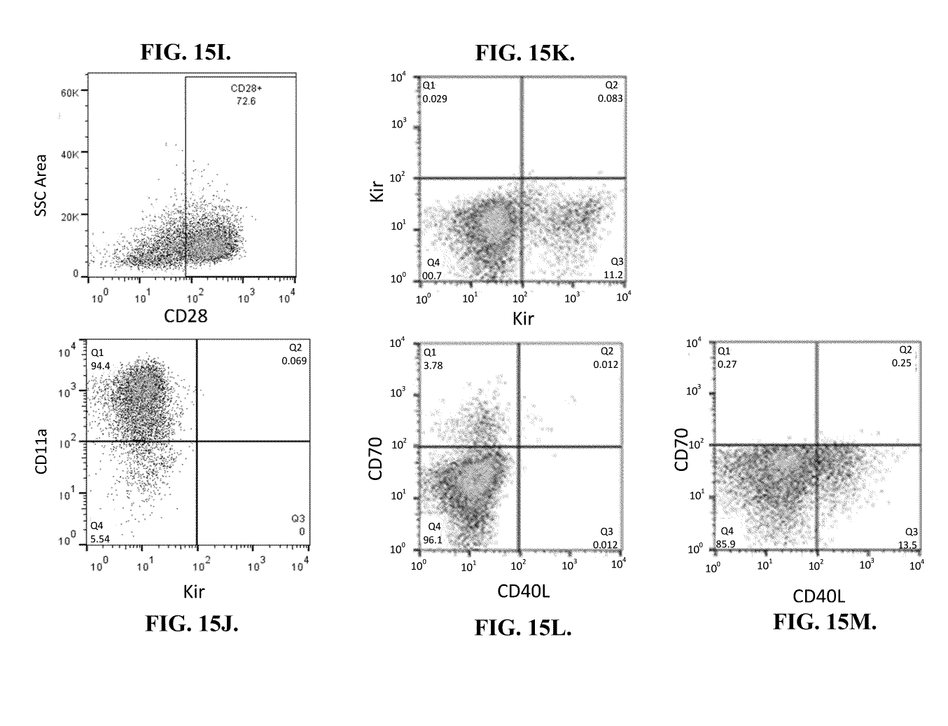

The expression of methylation-sensitive genes was analyzed in 5-azaC treated cells from healthy women. PBMCs from a healthy woman were stimulated with PHA then cultured with or without 5-azaC. Seventy-two hours later the cells were washed, stained with fluorochromeconjugated antibodies to CD3, CD4, CD28, CD11a, CD70 and CD40L with and without a “cocktail” of PE conjugated antibodies to 5 distinct KIR proteins then analyzed by flow cytometry as outlined in FIG. 10. An example of the gating strategy used to select the T cell subsets and establish negative and positive staining for each antibody is shown in Supplemental FIGS. 15A-M. Doublets were excluded based on forward and side scatter (FIGS. 15A-C), thus assuring analysis of single cells. Greater than 95% of CD3⁺CD4⁺ T cells from healthy donors were CD28⁺ (FIG. 15E) a value typical for young healthy donors although this percent declines somewhat with age (ref 26; herein incorporated by reference in its entirety). FIG. 15F shows CD11a and KIR expression on the CD3⁺CD4⁺CD28⁺ T cells selected in FIG. 15E. CD3⁺CD4⁺CD28⁺KIR⁺ T cells were heterogeneous in their KIR expression. Since a cocktail of 5 antibodies directed to different KIR gene products was used to provide the broadest coverage of KIR expression, the heterogeneity observed may reflect different clones, different amounts of a given KIR isotype on the cells or heterogeneity in the assortment of KIR genes inherited by any given person (ref. 27; herein incorporated by reference in its entirety).

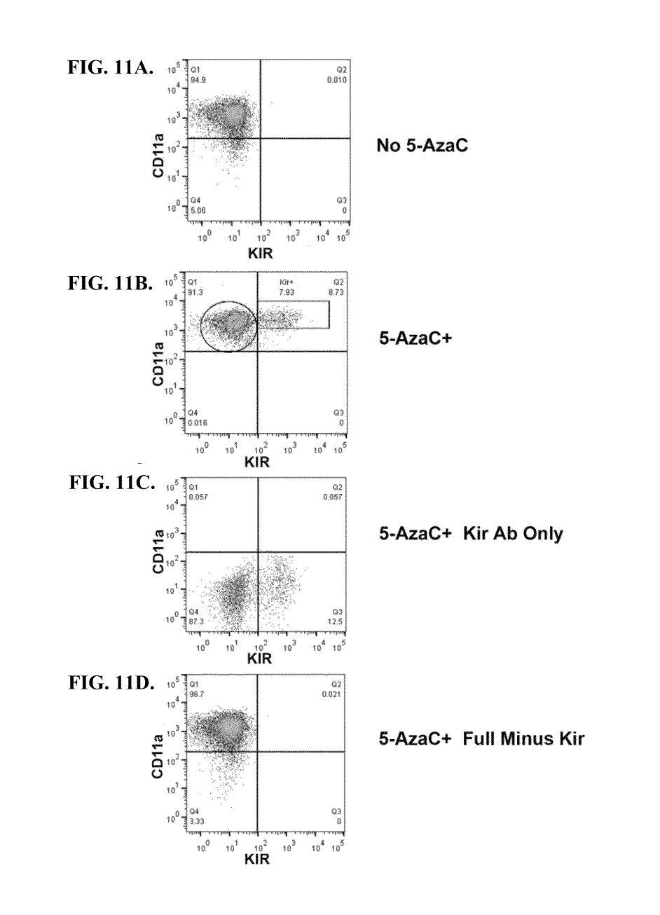

FIGS. 11A-D shows KIR and CD11a expression on untreated and 5-azaC treated T cells. In the absence of treatment with the demethylating agent 5-azaC very few KIR+ cells were observed (FIG. 11A). The small number of KIR⁺ cells observed in the peripheral blood of healthy people varies and increases somewhat with age (ref. 28; herein incorporated by reference in its entirety). Treatment with 5-azaC increases the numbers of KIR+ cells (FIG. 11B) (ref. 29; herein incorporated by reference in its entirety). Further, CD11a expression on the majority (>70%) of the KIR⁺CD3⁺CD4⁺CD28⁺ cells treated with 5-azaC (FIG. 11B, rectangle) was nearly twice that of the corresponding KIR− T cells (FIG. 11B, circle) from the same cultures (median fluorescence intensity 2298+/−748 SD, versus 1394+/−572, respectively, n=9 analyzed in 7 different experiments, p<0.001). Therefore, the KIR⁺ subset was referred to as CD3⁺CD4⁺CD28⁺CD11a^{hi}KIR⁺. While CD4⁺ T cells express CD11a, overexpression of CD11a confers autoreactivity on CD4⁺ T cells (ref 11; herein incorporated by reference in its entirety). Single antibody (PE-KIRs) and full minus one (FMO, full staining minus PE-KIRs) staining demonstrated that the KIR staining was specific (FIGS. 11C and 11D, respectively).

### Example 5

### Epigenetically Altered T Cells in Autoimmune Diseases

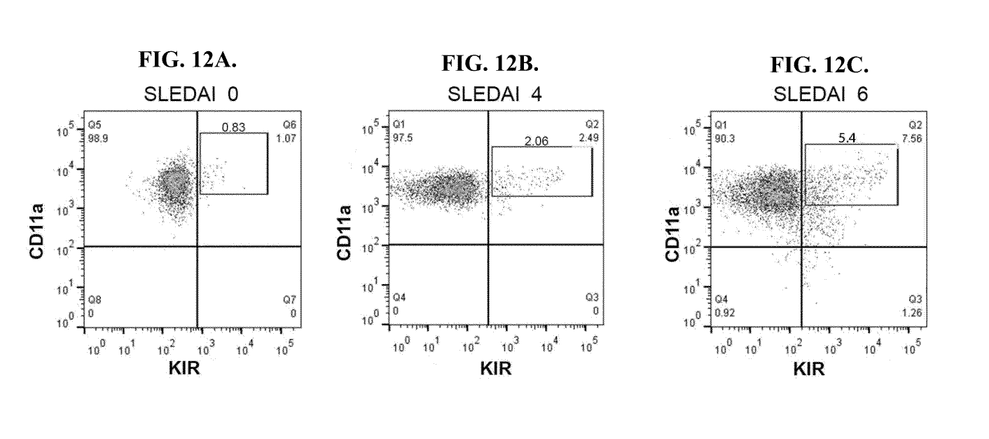

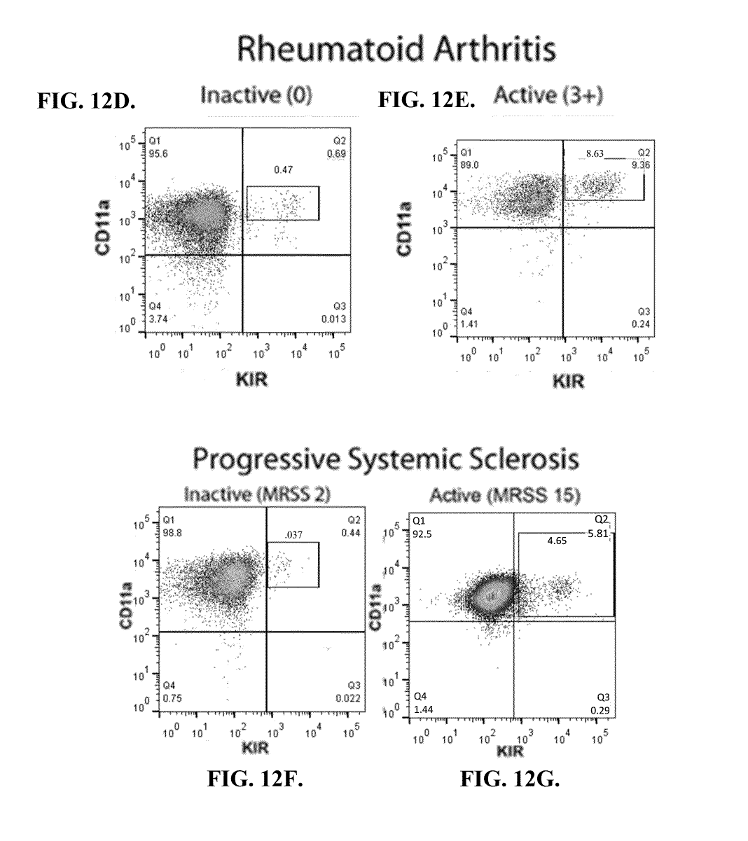

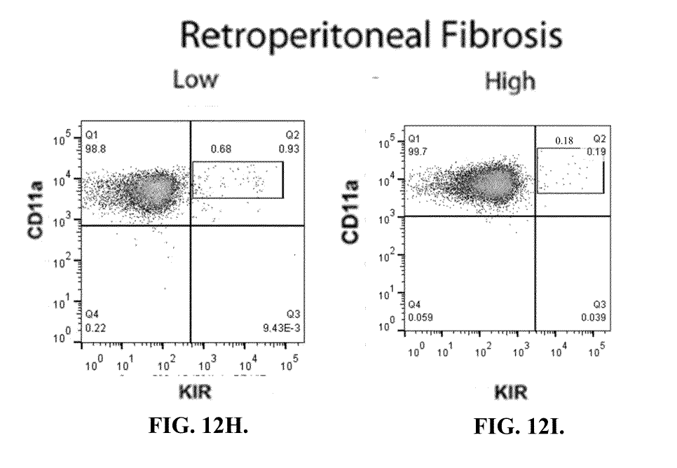

Experiments were conducted during development of embodiments to determine whether women with active lupus had a similar CD3⁺CD4⁺CD28⁺CD11a^{hi}KIR⁺ subset. FIG. 12A shows an example of CD11a and KIR expression on CD3⁺CD4⁺CD28⁺ T cells from a patient with inactive SLE. Less than 1% of CD3⁺CD4⁺CD28⁺ cells were also KIR⁺ (rectangle). However, the percent of CD3⁺CD4⁺CD28₊KIR⁺ cells increased with SLE disease activity (FIGS. 12B-C). The KIR⁺ T cells also overexpressed CD11a. Evidence for the CD3⁺CD4₊CD28⁺CD11a^{hi}KIR⁺ T cell subset in other systemic autoimmune rheumatic diseases was tested in patients with active and inactive RA and active and inactive SSc. Negative controls included T cells from patients with RPF, an idiopathic inflammatory fibrosing disorder classified in the IgG4-related sclerosing disease spectrum 30 and healthy age-matched donors. Background levels of CD3⁺CD4⁺CD28⁺CD11a^{hi}KIR⁺ T cells were observed in RA and SSc patients with inactive or mild disease (FIGS. 12D,F). As in the SLE patients, the size of the KIR⁺ subset was larger in patients with active RA (FIG. 12E) and more extensive SSc (FIG. 12G) and expressed more CD11a protein than the KIR⁻ cells. In contrast the size of the KIR⁺ subset in RPF patients was comparable to that of the CD3⁺CD4⁺CD28⁺KIR⁺ subset in healthy controls and did not increase with disease activity (FIGS. 12H,I).

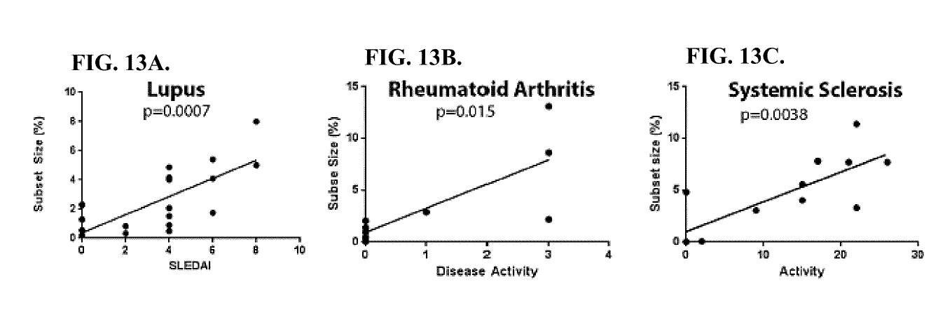

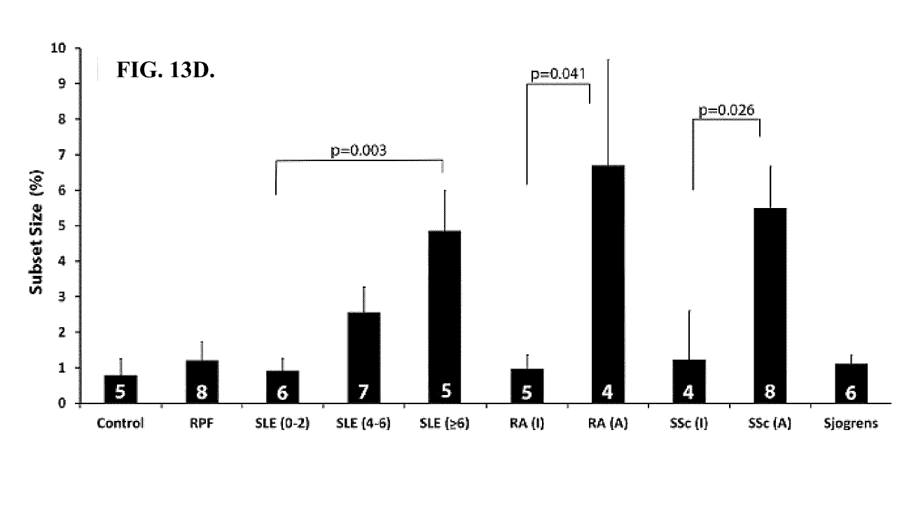

FIGS. 13A-D shows there was a direct relationship between the subset size and the SLEDAI (n=18, p=0.0007 by linear regression, FIG. 13A). Similarly the relationship between the size of the KIR⁺ subset and RA and SSc disease activity was highly significant (FIGS. 13B,C). The subset was seen in 2 of the 6 patients with Sjogren's Syndrome; however, 4 of these subjects had a EULAR Sjogren's Syndrome disease activity index (ref. 31; herein incorporated by reference in its entirety) of 0 and two had a score of 1, with no relation of the score to subset size. The subset was not seen in 8 patients with RPF, two of whom were judged to have active disease as determined by the clinical criteria (ref 24; herein incorporated by reference in its entirety).

### Example 6

### Co-Expression of Other Methylation-Sensitive Genes

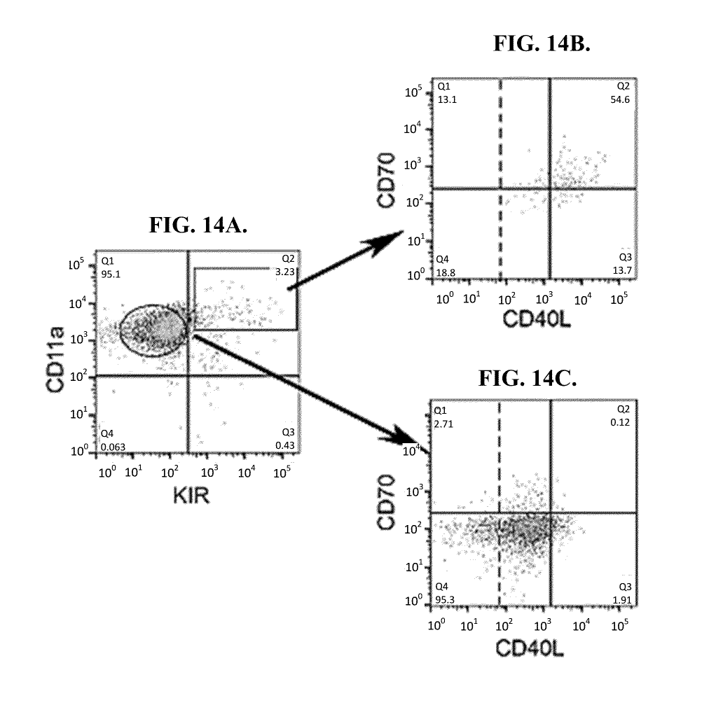

Data from experiments were conducted during development of embodiments herein demonstrates that the CD4⁺ T cells from patients with active lupus-related autoimmune diseases express elevated levels of CD11a and KIR and that the percent of the KIR⁺ cells correlates with disease activity. Further experiments were conducted to determine whether the methylation-sensitive genes CD70 and CD40L were also expressed on the KIR⁺ T cells and to what level they were co-expressed on KIR⁻ T cells. Studies of CD40L activation were confined to women because CD40L is encoded on the X chromosome, one of which is inactivated by DNA methylation in women, so female CD4⁺ T cells demethylated with 5-azaC or in women with active SLE overexpress CD40L while CD40L expression is not affected in male T cells (ref. 32; herein incorporated by reference in its entirety). An example of co-expression in a woman with active SLE is shown in FIGS. 14A-C. Nearly all CD4⁺ T cells also express CD40L (FIGS. 14B-C, dotted line). However, with experimentally induced DNA demethylation and in patients with active SLE, CD40L expression is elevated (ref 32; herein incorporated by reference in its entirety). In women with active SLE (SLEDAI 4-8), the majority of the CD4⁺CD28₊CD11a^{hi}KIR⁺CD40L^{hi} cells were also CD70⁺ (Mean 76+/−8 SEM, range 45-95%, n=6). In women with active RA 70+/−8% SEM, (n=5 range 55-94%) of their CD4⁺CD28⁺CD11a^{hi}KIR⁺CD40L^{hi} T cells also over-expressed CD70. Similar results (67%+/−10%, n=5) were observed in women with more extensive SSc (MRSS >9, not shown). In contrast, 3% or less of the CD4⁺CD28⁺CD11a⁺KIR⁻ T cells from patients with SLE overexpressed CD40L but those cells did not express CD70. A small number (<3%) of CD4⁺CD28⁺CD11a⁺KIR⁻ cells expressed CD70 but did not exhibit elevated levels of CD40L (FIG. 14C). Similar results were observed in patients with RA and SSc.

### Example 7

### Exemplary Methods

### Subjects

Patients with SLE, RA, SSc, Sjögren's syndrome and RPF were recruited from the rheumatology and nephrology outpatient clinics at the University of Michigan. Healthy controls, ages 21-64 years, were recruited by advertising. Patients with Sjögren's syndrome met criteria for the classification of primary Sjogren's Syndrome (ref 17; herein incorporated by reference in its entirety), and disease activity was determined using the EULAR Sjogren's syndrome disease activity index (ESSDAI) (ref. 18; herein incorporated by reference in its entirety). Patients with lupus met the American College of Rheumatology classification criteria for systemic lupus erythematosus (refs 19-20; herein incorporated by reference in their entireties), and disease activity was determined by SLEDAI (ref. 8; herein incorporated by reference in its entirety). Patients with RA met the 2010 criteria for the classification of rheumatoid arthritis (ref. 21; herein incorporated by reference in its entirety). RA disease activity was assessed on a qualitative 0-4 scale with 1 point given for each of the following criteria: 1) provider added an immunosuppressant or increased the dose of a currently prescribed immunosuppressant during the visit, 2) patient reported morning stiffness >30 minutes, 3) provider documented synovitis was present on exam, 4) ESR or CRP above normal or increased from prior visit when judged to be inactive.

Patients receiving cyclophosphamide were excluded because of effects on T cell surface marker expression (ref 22; herein incorporated by reference in its entirety). SSc patients were further classified as sine scleroderma (no skin involvement), limited cutaneous SSc, or diffuse cutaneous SSc based on skin examination and extent of involvement assessed using the modified Rodnan skin score (MRSS) (ref 23; herein incorporated by reference in its entirety). Only subjects with idiopathic RPF were included. The RPF diagnosis was confirmed by expert opinion of a rheumatologist and nephrologist, as no standardized classification criteria for RPF currently exists. The diagnosis is suspected by clinical presentation with back pain and ureteral obstruction associated with characteristic imaging abnormalities, elevated in inflammatory markers, and tissue revealing fibrotic and non-specific lymphocytic infiltrates negative for malignancy (ref. 24; herein incorporated by reference in its entirety). The majority of RPF patients had characteristic CT imaging and ureteral obstruction; all but one had biopsies performed during workup.

### T Cell Isolation and Culture

Peripheral blood mononuclear cells (PBMC) were isolated from the venous blood of patients or healthy controls by Ficoll density gradient separation (Histopaque-1077, Sigma Aldrich, St. Louis, Mo.). Where indicated PBMC from healthy female donors were stimulated with 1 μg/ml phytohemagglutinin (Remel, Lenexa, Kans.) for 18-24 hr at 37° C., in a 5% CO2/balanced air environment, followed by treatment with 2.5 μM 5-azacytidine (5-azaC), (Sigma Chemical Co., St. Louis, Mo.) for 3 days (ref 25; herein incorporated by reference in its entirety).

### Antibodies and Flow Cytometric Analyses

The following antibodies were used: PE-anti-Kir2DL4/CD158D (clone 181703; R&D Systems, Minneapolis, Minn.), anti-CD40L-biotin (clone hCD40L-M91) and avidin-PECy7, PE-Cy7-anti-CD8 (clone RPA-T8), anti-CD11a-APC (clone HI111), Pacific Blue-anti-CD3 (clone UCHT1), PECy5-anti-CD28 (clone CD28.2), FITC-anti-CD70 (clone Ki-24), and APC-Cy7-anti-CD4 (clone RPAT4) (Becton Dickinson, Franklin Lakes, N.J.). PE-anti-CD158b (clone CH-L), PE-anti-CD158i (clone FES172), PE-anti-CD158b1/b2, j (clone GL183), and PE-anti-CD158a,h (clone EB6B) were from Beckman Coulter (Brea, Calif.). Antibodies were titrated to determine their optimal concentrations prior to use.

PBMC from patients with autoimmune diseases or from cultures of 5-azaC treated cells were incubated in PBS/0.001% azide containing 10% horse serum at 4° C. for 30 min to block nonspecific binding. Staining procedures were performed at 4° C. in the dark. The cells were then washed, incubated for 60 min. with a single or mixture of fluorochrome-conjugated antibodies. Biotin-CD40L stained cells were further washed and incubated for 30 minutes with a 1:1000 dilution of avidin-PE-Cy7, fixed with 4% paraformaldehyde and stored in PBS/0.001% azide in the dark at 4° C. until analyzed. Controls included isotype and fluorochrome matched antibodies, single antibodies, and “full minus one” (FMO) staining controls in which one of each of the fluorochrome-conjugated antibodies was serially omitted while retaining the rest.

FACS analyses were performed using a FACS ARIA IIIU flow cytometer and FACSDiva software version 6.1.3 (Becton Dickinson, N.J.) or an iCyte Synergy (Sony Biotechnology, San Jose Calif.) and WINLIST version 8 (Verty Software House) software. The fluorescent antibody conjugates, laser emission wavelengths and filter sets used are shown in Table 1.

### Statistical Analysis

The difference between two means was evaluated with Student's t-test, and between subset size and disease activity by regression analysis using GraphPad Prism 6 software. The median and standard deviations (SD) of populations identified by flow cytometry were calculated using the statistical functions within the FlowJo software (FlowJo LLC, Ashland Oreg.).

TABLE 1
Flow cytometry instrument configuration.

| | | Laser Emission | | |
|---|---|---|---|---|
| | | Wavelength | Filter | Filter |
| Antibody | Fluorochrome | (nm) | (ARIA IIIU) | (SYNERGY) |
| CD3 | Pacific-Blue | 405 | 450/40 | 455/50 |
| CD70 | FITC | 488 | 530/30 | 525/50 |
| CD28 | PE-CY5 | 488 | 695/40 | 665/30 |
| CD28 | PE-CY5 | 561 | 670/14 | |
| CD40L | PE-Cy7 | 561 | 780/60 | 775/50 |
| Kir | PE | 561 | 582/15 | 577/25 |
| CD11a | APC | 633 | 660/20 | 665/30 |
| CD11a | APC | R/O (594/685) | | 665/30 |
| CD4 | APC-Cy7 | 633 | 780/60 | |
| CD4 | APC-Cy7 | R/O (594/685) | | 775/50 |

### Example 8

Experiments conducted during development of embodiments herein demonstrate that the surface markers identified herein to be present in/on the CD3⁺CD4⁺CD28⁺CD11a^{hi}CD70⁺CD40L^{hi}KIR⁺ T lymphocyte subset are also present in/on a CD3⁺CD4⁺CD28⁻CD11a^{hi}CD70⁺CD40L^{hi}KIR⁺ T lymphocytes. It has been established in the field that CD4⁺CD28⁻ T cells are involved in atherosclerosis. These CD4⁺CD28⁻ T cells are a senescent subset which invade atherosclerotic plaques, causing further plaque development and rupture. It is also known in the field that patients with lupus, as well as the related autoimmune diseases, develop rapidly progressive atherosclerosis; although the cause has not been established in the literature. Experiments conducted during development of embodiments herein indicate that the CD3⁺CD4⁺CD28⁺CD11a^{hi}CD70⁺CD40L^{hi}KIR⁺ T lymphocyte subset present in autoimmune diseases senesce in CD3⁺CD4⁺CD28⁻CD11a^{hi}CD70⁺CD40L^{hi}KIR⁺ T lymphocytes which participate in plaque development and rupture. Such a mechanism indicates that targeting of the CD3⁺CD4⁺CD28⁺CD11a^{hi}CD70⁺CD40L^{hi}KIR⁺ T lymphocyte subset will prevent formation of the CD3⁺CD4⁺CD28ⁱCD11a^{hi}CD70⁺CD40L^{hi}KIR⁺ T lymphocytes and the subsequence atherosclerotic consequences.

### REFERENCES

The following references, some of which are cited above by author name or number, are herein incorporated by reference in their entireties.
1. Palacios R, Santos J, Valdivielso P, et al. Human immunodeficiency virus infection and systemic lupus erythematosus. An unusual case and a review of the literature. Lupus 2002; 11(1):60-3.
2. Fox R A, Isenberg D A. Human immunodeficiency virus infection in systemic lupus erythematosus. Arthritis Rheum 1997; 40(6):1168-72.
3. Wofsy D, Seaman W E. Successful treatment of autoimmunity in NZB/NZW F1 mice with monoclonal antibody to L3T4. The Journal of experimental medicine 1985; 161(2):378-91.
4. Jabs D A, Burek C L, Hu Q, et al. Anti-CD4 monoclonal antibody therapy suppresses autoimmune disease in MRL/Mp-lpr/lpr mice. Cell Immunol 1992; 141(2):496-507.
5. Richardson B. Effect of an inhibitor of DNA methylation on T cells. II. 5-Azacytidine induces self-reactivity in antigen-specific T4+ cells. Human immunology 1986; 17(4):456-70.
6. Patel D R, Richardson B C. Dissecting complex epigenetic alterations in human lupus. Arthritis research & therapy 2013; 15(1):201.
7. Richardson B. Primer: epigenetics of autoimmunity. Nature clinical practice Rheumatology 2007; 3(9):521-7.
8. Bombardier C, Gladman D D, Urowitz M B, et al. Derivation of the SLEDAI. A disease activity index for lupus patients. The Committee on Prognosis Studies in SLE. Arthritis and rheumatism 1992; 35(6):630-40.
9. Somers E C, Thomas S L, Smeeth L, et al. Autoimmune diseases co-occurring within individuals and within families: a systematic review. Epidemiology 2006; 17(2):202-17.
10. Somers E C, Thomas S L, Smeeth L, et al. Are individuals with an autoimmune disease at higher risk of a second autoimmune disorder? American journal of epidemiology 2009; 169(6):749-55.
11. Cooper G S, Bynum M L, Somers E C. Recent insights in the epidemiology of autoimmune diseases: improved prevalence estimates and understanding of clustering of diseases. Journal of autoimmunity 2009; 33(3-4):197-207.
12. Anaya J M, Gomez L, Castiblanco J. Is there a common genetic basis for autoimmune diseases? Clinical & developmental immunology 2006; 13(2-4):185-95.
13. Ramos P S, Criswell L A, Moser K L, et al. A comprehensive analysis of shared loci between systemic lupus erythematosus (SLE) and sixteen autoimmune diseases reveals limited genetic overlap. PLoS genetics 2011; 7(12):e1002406.
14. Somers E C, Ganser M A, Warren J S, et al. Mercury Exposure and Antinuclear Antibodies among Females of Reproductive Age in the United States: NHANES. Environmental health perspectives 2015; 123(8):792-8.
15. Kubo K, Yamamoto K. IgG4-related disease. International journal of rheumatic diseases 2015.
16. Urban M L, Palmisano A, Nicastro M, et al. Idiopathic and secondary forms of retroperitoneal fibrosis: a diagnostic approach. La Revue de medecine interne/fondee par la Societe nationale francaise de medecine interne 2015; 36(1):15-21.
17. Vitali C, Bombardieri S, Jonsson R, et al. Classification criteria for Sjogren's syndrome: a revised version of the European criteria proposed by the American-European Consensus Group. Annals of the rheumatic diseases 2002; 61(6):554-8.
18. Seror R, Ravaud P, Bowman S J, et al. EULAR Sjogren's syndrome disease activity index: development of a consensus systemic disease activity index for primary Sjogren's syndrome. Annals of the rheumatic diseases 2010; 69(6):1103-9.
19. Tan E M, Cohen A S, Fries J F, et al. The 1982 revised criteria for the classification of systemic lupus erythematosus. Arthritis and rheumatism 1982; 25(11):1271-7.
20. Hochberg M C. Updating the American College of Rheumatology revised criteria for the classification of systemic lupus erythematosus. Arthritis and rheumatism 1997; 40(9):1725.
21. Aletaha D, Neogi T, Silman A J, et al. 2010 Rheumatoid arthritis classification criteria: an American College of Rheumatology/European League Against Rheumatism collaborative initiative. Arthritis and rheumatism 2010; 62(9):2569-81.
22. McCune W J, Golbus J, Zeldes W, et al. Clinical and immunologic effects of monthly administration of intravenous cyclophosphamide in severe systemic lupus erythematosus. N Engl J Med 1988; 318(22):1423-31.
23. Khanna D, Denton C P. Evidence-based management of rapidly progressing systemic sclerosis. Best practice & research Clinical rheumatology 2010; 24(3):387-400.
24. Swartz R D. Idiopathic retroperitoneal fibrosis: a review of the pathogenesis and approaches to treatment. American journal of kidney diseases: the official journal of the National Kidney Foundation 2009; 54(3):546-53.
25. Liu Y, Chen Y, Richardson B. Decreased DNA methyltransferase levels contribute to abnormal gene expression in “senescent” CD4(+)CD28(−) T cells. Clinical immunology 2009; 132(2):257-65.
26. Weyand C M, Brandes J C, Schmidt D, et al. Functional properties of CD4+CD28− T cells in the aging immune system. Mechanisms of ageing and development 1998; 102(2-3):131-47.
27. Middleton D, Gonzelez F. The extensive polymorphism of KIR genes. Immunology 2010; 129(1):8-19.
28. Li Y, Liu Y, Strickland F M, et al. Age-dependent decreases in DNA methyltransferase levels and low transmethylation micronutrient levels synergize to promote overexpression of genes implicated in autoimmunity and acute coronary syndromes. Exp Gerontol 2010; 45(4):312-22.
29. Liu Y, Kuick R, Hanash S, et al. DNA methylation inhibition increases T cell KIR expression through effects on both promoter methylation and transcription factors. Clinical immunology 2009; 130(2):213-24.
30. Brito-Zeron P, Ramos-Casals M, Bosch X, et al. The clinical spectrum of IgG4-related disease. Autoimmunity reviews 2014; 13(12):1203-10.
31. Seror R, Bootsma H, Saraux A, et al. Defining disease activity states and clinically meaningful improvement in primary Sjogren's syndrome with EULAR primary Sjogren's syndrome disease activity (ESSDAI) and patient-reported indexes (ESSPRI). Annals of the rheumatic diseases 2014.
32. Lu Q, Wu A, Tesmer L, et al. Demethylation of CD40LG on the inactive X in T cells from women with lupus. Journal of immunology 2007; 179(9):6352-8.
33. Farrar W L, Ruscetti F W, Young H A. 5-Azacytidine treatment of a murine cytotoxic T cell line alters interferon-gamma gene induction by interleukin 2. Journal of immunology 1985; 135(3):1551-4.
34. Yung R L, Quddus J, Chrisp C E, et al. Mechanism of drug-induced lupus. I. Cloned Th2 cells modified with DNA methylation inhibitors in vitro cause autoimmunity in vivo. Journal of immunology 1995; 154(6):3025-35.
35. Li Y, Gorelik G, Strickland F M, et al. Oxidative stress, T cell DNA methylation, and lupus. Arthritis & rheumatology 2014; 66(6):1574-82.
36. Strickland F M, Li Y, Johnson K, et al. CD4(+) T cells epigenetically modified by oxidative stress cause lupus-like autoimmunity in mice. Journal of autoimmunity 2015; 62:75-80.
37. Driscoll T, Jacklyn G, Orchard J, et al. The global burden of occupationally related low back pain: estimates from the Global Burden of Disease 2010 study. Annals of the rheumatic diseases 2014; 73(6):975-81.
38. Gorelik G, Sawalha A H, Patel D, et al. T cell PKCdelta kinase inactivation induces lupus-like autoimmunity in mice. Clinical immunology 2015; 158(2):193-203.
39. Perl A. Oxidative stress in the pathology and treatment of systemic lupus erythematosus. Nature reviews Rheumatology 2013; 9(11):674-86.
40. Pals S T, Radaszkiewicz T, Roozendaal L, et al. Chronic progressive polyarthritis and other symptoms of collagen vascular disease induced by graft-vs-host reaction. Journal of immunology 1985; 134(3):1475-82.
41. Daikeler T, Tyndall A. Autoimmunity following haematopoietic stem-cell transplantation. Best practice & research Clinical haematology 2007; 20(2):349-60.
42. Minhas U, Das P, Bhatnagar A. Role of reactive intermediates in the immunopathogenesis of the pristane-induced Balb/c model of lupus. Lupus 2011; 20(13):1421-5.
43. Satoh M, Hamilton K J, Ajmani A K, et al. Autoantibodies to ribosomal P antigens with immune complex glomerulonephritis in SJL mice treated with pristane. Journal of immunology 1996; 157(7):3200-6.
44. Stasiuk L M, Ghoraishian M, Elson C J, et al. Pristane-induced arthritis is CD4+ T-cell dependent. Immunology 1997; 90(1):81-6.
45. Leiss H, Niederreiter B, Bandur T, et al. Pristane-induced lupus as a model of human lupus arthritis: evolvement of autoantibodies, internal organ and joint inflammation. Lupus 2013; 22(8):778-92.
46. Odobasic D, Muljadi R C, O'Sullivan K M, et al. Suppression of Autoimmunity and Renal Disease in Pristane-Induced Lupus by Myeloperoxidase. Arthritis & rheumatology 2015; 67(7):1868-80.
47. Li J, Wu H, Huang X, et al. Clinical analysis of 56 patients with rhupus syndrome: manifestations and comparisons with systemic lupus erythematosus: a retrospective case-control study. Medicine 2014; 93(10):e49.
48. Mosca M, Tani C, Vagnani S, et al. The diagnosis and classification of undifferentiated connective tissue diseases. Journal of autoimmunity 2014; 48-49:50-2.
49. Kuo C F, Grainge M J, Valdes A M, et al. Familial Aggregation of Systemic Lupus Erythematosus and Coaggregation of Autoimmune Diseases in Affected Families. JAMA internal medicine 2015.

1. A method of treating an autoimmune disease and/or atherosclerosis comprising administering to a subject an agent that selectively targets CD3+CD4+CD28+CD11ahiCD70+CD40LhiKIR+ T lymphocytes in the subject.

2. The method of claim 1, wherein the agent comprises an antibody or antibody fragment.

3. The method of claim 1, wherein the agent binds an epitope presented on the surface of the CD3+CD4+CD28+CD11ahiCD70+CD40LhiKIR+ T lymphocytes.

4. The method of claim 3, wherein the epitope is presented on the surface of at least 75% of the CD3+CD4+CD28+CD11ahiCD70+CD40LhiKIR+ T lymphocytes in the subject or in subjects with active lupus.

5. The method of claim 4, wherein the epitope is presented on the surface of less than 20% of NK cells in the subject or in subjects with active lupus.

6. The method of claim 1, wherein the agent binds one or more MR proteins present on the CD3+CD4+CD28+CD11ahiCD70+CD40LhiKIR+ T lymphocytes.

7. The method of claim 6, wherein the agent binds one or more MR protein selected from the group consisting of KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1.

8. The method of claim 7, wherein the agent binds only one of KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1.

9. The method of claim 1, wherein the agent is multispecific, recognizing two or more epitopes presented on the CD3+CD4+CD28+CD11ahiCD70+CD40LhiKIR+ T lymphocytes.

10. The method of claim 9, wherein the agent binds at least one MR protein selected from the group consisting of KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1.

11. The com method position of claim 10, wherein agent binds at least two MR proteins selected from the group consisting of KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1.

12. The method of claim 1, wherein the agent a conjugate with a molecular agent configured to kill a cell to which the agent binds.

13. The method of claim 12, wherein the molecular agent is selected from the selected from the group consisting of small molecule drugs, toxins, peptides, polypeptides, and antibodies.

14. The method of claim 1, wherein the agent is coadministered with one or more additional treatments for the autoimmune disease.

15. The method of claim 14, wherein the additional treatments are selected from immunosuppressives and anti-inflammatories.

16. The method of claim 1, wherein the autoimmune disease is selected from the group consisting of systemic lupus erythematosus (SLE), rheumatoid arthritis, Sjogren's syndrome, progressive systemic sclerosis (PSS), multiple sclerosis.

17. The method of claim 1, wherein the autoimmune disease is SLE.

18.-19. (canceled)

20. The method of claim 1, wherein the subject suffers from an autoimmune disease selected from the group consisting of systemic lupus erythematosus (SLE), rheumatoid arthritis, Sjogren's syndrome, progressive systemic sclerosis (PSS), multiple sclerosis.

21. The method of claim 1, wherein the subject suffers from atherosclerosis but does not suffer from an autoimmune disease selected from the group consisting of systemic lupus erythematosus (SLE), rheumatoid arthritis, Sjogren's syndrome, progressive systemic sclerosis (PSS), multiple sclerosis.

22.-32. (canceled)

33. The method of claim 1, wherein the agent is coadministered with one or more additional treatments for atherosclerosis.
